(19) United States
(12) Patent Application Publication
(10) Pub. No.: US 2024/0425940 A1
(43) Pub. Date: Dec. 26, 2024

(54) COMPOSITIONS AND METHODS FOR ISOTHERMAL AMPLIFICATION OF NUCLEIC ACIDS

(71) Applicant: Cue Health Inc., San Diego, CA (US)

(72) Inventor: Ayub Khattak, San Diego, CA (US)

(21) Appl. No.: 18/757,285

(22) Filed: Jun. 27, 2024

Related U.S. Application Data
(63) Continuation of application No. PCT/US2021/065780, filed on Dec. 30, 2021.

(51) Int. Cl. C12Q 1/70; C12Q 1/6844; C12Q 1/6876

(52) U.S. Cl. CPC C12Q 1/701; C12Q 1/6844; C12Q 1/6876

(57) ABSTRACT

The present disclosure relates generally to recombinant nucleic acid primers (oligonucleotide) comprising a hairpin structure and a methylated RNA region, and nucleic acid amplification processes comprising the recombinant nucleic acid primers for the isothermal amplification of target nucleic acids with higher specificity and sensitivity in single or multiplexed assays. Further provided are compositions comprising the recombinant nucleic acid primers, and devices suitable for use in or with the nucleic acid amplification processes.

### CROSS-REFERENCE TO RELATED APPLICATIONS

This application is a continuation of International Patent Application No. PCT/US2021/065780, filed Dec. 30, 2021, the content of which is herein incorporated by reference in its entirety for all purposes.

### TECHNICAL FIELD

The present disclosure relates to recombinant nucleic acid primers, compositions, methods and devices for isothermal amplification of target nucleic acids for multiplexed assays.

### BACKGROUND

The following description of the background of the present technology is provided simply as an aid in understanding the present technology and is not admitted to describe or constitute prior art to the present technology.

Efficient amplification of nucleic acids is an important part of clinical diagnostic and medical research. In particular, amplification of nucleic acid is important to detect the presence or absence of pathogens, disease, or condition within a human or other animals. Nucleic acid amplification as a diagnostic tool started with the inception of polymerase chain reaction (PCR) technology, and expanded with the recent advances in whole genome sequencing and identification of biological markers for diseases.

PCR is a very sensitive and efficient method of amplifying short nucleic acid sequences contained within larger and more complex nucleic acid molecules. However, PCR is limited because it requires thermocycling the amplification reaction at very high temperatures. In particular, PCR requires high temperatures to: (1) melt (i.e, denature) the target double stranded DNA (>90° C.) to generate two single-stranded DNA molecules; (2) facilitate primers hybridization (i.e., annealing, 50-60° C.) to the denatured single stranded DNA; and (3) polymerize/amplify the primers (72° C.) to generate a new copy of the single strand nucleic acid. Indeed, PCR uses thermostable polymerases that can only function at extremely high temperatures (72° C.).

PCR is further limited by the cost of the sophisticated machines that are required to smoothly and efficiently change the temperatures during the amplification process. PCR amplification techniques require expensive laboratory equipments and the expertise of highly-trained medical professionals. Consequently, diagnostic methods that rely on PCR techniques are typically performed within laboratories or medical facilities. As such many medical conditions may go undiagnosed because of the lack of accessibility to a research laboratory or a medical facility. Moreover, infectious diseases spread and cause substantial harm to a population before the pathogenic agent is identified.

To make nucleic acid amplification techniques more accessible as diagnostics tools, a second class of amplification techniques, known as isothermal techniques, were recently developed. Recently developed isothermal amplification techniques include, but are not limited to: Loop-mediated isothermal amplification (LAMP or Loopamp); strand displacement amplification (SDA); recombinase polymerase amplification (RPA); helicase dependent amplification (HDA); Isothermal Multiple Displacement Amplification (IMDA); Rolling Circle Amplification; polymerase spiral reaction (PSR); signal-mediated amplification of RNA technology (SMART); and nicking enzyme amplification reaction (NEAR). The isothermal amplification process does not require thermal melting of DNA or any thermostable components. Rather, isothermal amplification techniques are performed at a single and constant temperature, using the biological activity of enzymes rather than temperature changes to copy nucleic acid sequences. In particular, some isothermal amplification techniques rely on the biological activity of enzymes involved during homologous recombination (i.e., DNA repair, recombinases and accessory molecules), DNA replication (i.e., helicases and accessory molecules), or a restriction enzyme to melt the target double stranded DNA at a desired region (e.g., a nucleic acid region that is complementary to one or more oligonucleotide that is present in the amplification); specific polymerases (DNA and RNA polymerase; strand-displacing DNA polymerase); and/or specially designed primers (a single primer, primer pairs, multiple primer sets, or DNA-RNA chimeric primers).

As an emerging technology, isothermal amplification still has many drawbacks. For instance, many enzymes and their accessory molecules do not perform well in vitro. Some enzymes compete with their accessory molecules for primers binding, which reduces the kinetic of the amplification process. In addition, isothermal amplification products contain multiple amplification artifacts caused by non-specific interaction between different components of the isothermal amplification machinery. The presence of high level of artifactual amplification products reduces the sensitivity and specificity of the isothermal amplification assay in medical diagnostics. All these drawbacks amper the use of this nascent, yet exciting, technology in medical diagnostics as a single or multiplexed amplification assay.

Accordingly, there is a need for an alternative and/or improved isothermal nucleic acid amplification process with high specificity and/or sensitivity for multiplexed amplification assay.

### SUMMARY OF THE DISCLOSURE

The present disclosure is based on the engineering of a novel recombinant nucleic acid primer for use as a non-functional primer (oligonucleotide) that binds to a recombinase enzyme for the invasion of double stranded nucleic acid during isothermal amplification. The novel recombinant nucleic acid primer provides a highly specific and sensitive amplification process with reduced non-specific amplification products.

One aspect of the present disclosure provides a recombinant nucleic acid primer (also referred to herein as a primer, a IO primer, or an oligonucleotide) comprising or consisting of: a hairpin region; and a target binding region that is complementary to a sequence of a target double stranded nucleic acid. In some embodiments, the target binding region comprises or consists essentially of at least one modified nucleoside (i.e., a modified base) to prevent amplification of the recombinant nucleic acid primer by a polymerase. In one embodiment, the at least one modified nucleoside (base) of the target binding region is selected from 5-nitroindole; 8-oxo-2′-deoxyguanosine (8-oxo-dG); 8-oxo7,8-dihydro-2′-deoxyguanosine; 8-oxo-deoxyadenosine (8-oxo-dA); 5-hydroxymethyl deoxycytosine (5-hydroxymethyl-dC); 5′-dimethoxytrityl-N-benzoyl-3′-deoxyadenosine,2′-succinoyl-long chain alkylamino-CPG (3′-dA-CPG); 5′-dimethoxytrityl-N-dimethylformamidine-3′-deoxyguanosine,2′-succinoyl-long chain alkylamino-CPG (3′-dG-CPG); 5′-dimethoxytrityl-N-benzoyl-3′-deoxyCytosine,2′-succinoyl-long chain alkylamino-CPG (3′-dC-CPG); 5′-dimethoxytrityl-3′-deoxythymidine,2′-succinoyl-long chain alkylamino-CPG (3′-dT-CPG); 3′-deoxyadenosine; cytosine arabinoside; inverted dT; 2′-O-methyl RNA nucleotide; 2′-O-methyladenosine (2′-OMe-A); 2′-O-methylcytosine (2′-OMe-C); 2′-O-methylguanosine (2′-OMe-G); 2′-O-methyluridine (2′-OMe-U); inosine, or a modified base that blocks 5′ to 3′ polymerase amplification. In another embodiment, the at least one modified nucleoside of the target binding region is a di-deoxy-nucleotide selected from dideoxycytidine (ddC); 2′-3′-dideoxycytidine (2′,3′ dideoxy-C); 5′-Dimethoxytrityl-N-succinoyl-long chain alkylamino-CPG, 2′,3′-deoxycytosine (2′,3′-ddC-CPG); 2′-3′-dideoxyadenosine (2′,3′ dideoxy-A); or 5′-dimethoxytrityl-N-succinoyl-long chain alkylamino-CPG, 2′,3′-deoxyadnosine (2′,3′-ddA-CPG). In some embodiments, the target binding region comprises a base modified with a C3 spacer amidite (DMT-1,3-propanediol); a C3 spacer phosphoramidite; a C3 spacer; a C6 spacer; hexanediol; a triethylene glycol spacer (spacer 9); a 18-atom hexa-ethyleneglycol spacer (spacer 18); 1′,2′-Dideoxyribose (dSpacer); a C6 amine; a peptide nucleic acid (PNA); a locked nucleic acid (LNA); or a 1-Dimethoxytrityloxy-propanediol-3-succinoyl)-long chain alkylamino-CPG (3′-Spacer-C3-CPG). In some embodiments, the at least one modified base of the target binding region that inhibits amplification by the polymerase is located at one or more of a 3′-end; a 5′-end; or within the internal region of the target binding region. In some embodiments, the modified base is a 2′-O-Methyl RNA (2′OME) nucleotide; optionally, wherein a region comprising at least one 2′OME nucleotides is a methylated RNA region. In one aspect, a region comprising at least one 2′OME nucleotides is a methylated RNA region.

One aspect of the present disclosure provides a recombinant nucleic acid primer (oligonucleotide) comprising or consisting of: (i) a hairpin region; (ii) a first methylated RNA region that is complementary to a sequence of a target nucleic acid; (iii) a target binding region that is complementary to a sequence of the target nucleic acid; and (iv) a second methylated RNA region that is complementary to a sequence of the target nucleic acid. The target nucleic acid can be double stranded or single stranded, and can be DNA or RNA.

In some embodiments, the hairpin region of the recombinant nucleic acid primer comprises or consists of a 5′ hairpin loop region and a stem region. In some embodiments, the first methylated RNA or the second methylated region comprises or consists of 2′-O-methyl RNA (2′OME) nucleotides.

In some embodiments, the recombinant nucleic acid primer is not extended by a polymerase. In some embodiments, the recombinant nucleic acid primer is resistant to amplification by a polymerase. In some embodiments, the recombinant nucleic acid primer is not a template for amplification, thereby resistant to non-specific amplification caused by the activity of a forward and/or reverse primers binding.

In some embodiments, the target binding region of the recombinant nucleic acid primer comprises one or more additional nucleoside modification. In one embodiment, the one or more additional modification is selected from 5-nitroindole; 8-oxo-2′-deoxyguanosine (8-oxo-dG); 8-oxo7,8-dihydro-2′-deoxyguanosine; 8-oxo-deoxyadenosine (8-oxo-dA); 5-hydroxymethyl deoxycytosine (5-hydroxymethyl-dC); 5′-dimethoxytrityl-N-benzoyl-3′-deoxyadenosine,2′-succinoyl-long chain alkylamino-CPG (3′-dA-CPG); 5′-dimethoxytrityl-N-dimethylformamidine-3′-deoxyguanosine,2′-succinoyl-long chain alkylamino-CPG (3′-dG-CPG); 5′-dimethoxytrityl-N-benzoyl-3′-deoxyCytosine,2′-succinoyl-long chain alkylamino-CPG (3′-dC-CPG); 5′-dimethoxytrityl-3′-deoxythymidine,2′-succinoyl-long chain alkylamino-CPG (3′-dT-CPG); 3′-deoxyadenosine; cytosine arabinoside; inverted dT; inosine, or a modified base that blocks 5′ to 3′ polymerase amplification. In another embodiment, the one or more additional nucleoside modification is a di-deoxy-nucleotide selected from dideoxycytidine (ddC); 2′-3′-dideoxycytidine (2′,3′ dideoxy-C); 5′-Dimethoxytrityl-N-succinoyl-long chain alkylamino-CPG, 2′,3′-deoxycytosine (2′,3′-ddC-CPG); 2′-3′-dideoxyadenosine (2′,3′ dideoxy-A); or 5′-dimethoxytrityl-N-succinoyl-long chain alkylamino-CPG, 2′,3′-deoxyadnosine (2′,3′-ddA-CPG). Yet, in another embodiments, the one or more additional nucleoside modification is a nucleoside modified with a C3 spacer amidite (DMT-1,3-propanediol); a C3 spacer phosphoramidite; a C3 spacer; a C6 spacer; hexanediol; a triethylene glycol spacer (spacer 9); a 18-atom hexa-ethyleneglycol spacer (spacer 18); 1′,2′-Dideoxyribose (dSpacer); a C6 amine; a peptide nucleic acid (PNA); a locked nucleic acid (LNA); or a 1-Dimethoxytrityloxy-propanediol-3-succinoyl)-long chain alkylamino-CPG (3′-Spacer-C3-CPG).

In some embodiments, the recombinant nucleic acid primer is used in a multiplexed assay. In some embodiments, the combination of the 5′ hairpin region and the first methylated RNA region significantly enhances the specificity and sensitivity of a multiplexed assay when compared to a recombinant nucleic acid primer comprising only the 5′ hairpin structure, the first methylated RNA region, or the second methylated RNA region. The specificity and sensitivity of multiplexed assay can be determined by, or is based on the occurrence of false positive results of the assay using prior art primers.

One aspect of the present disclosure provides a recombinant nucleic acid primer comprising or consisting of: (i) a 5′ hairpin region comprising or consisting of: a 5′ hairpin loop region that is about 25 to about 65 nucleotides in length; and stem region comprising a polydeoxycytidylic acid (poly-dC) region and/or having at least about 7-13 contiguous dC bases; (ii) a first methylated RNA region that is complementary to a sequence of a target nucleic acid comprising at least about one to at least about five 2′-methyl RNA (2′OME) nucleotides; (iii) a target binding region that is complementary to a sequence of the target nucleic acid, and (iv) a second methylated RNA region that is complementary to a sequence of the target nucleic acid comprising at least about 7-13 2′OME nucleotides. In some embodiments, the recombinant nucleic acid primer is not a template for amplification. In some embodiments, the recombinant nucleic acid primer significantly enhanced the specificity and sensitivity of a multiplexed assay when compared to a recombinant nucleic acid primer comprising only the 5′ hairpin structure, the first methylated RNA region, or the second methylated RNA region. The target nucleic acid can be double stranded or single stranded, and can be DNA or RNA.

The target nucleic acid can be DNA or RNA. In some embodiments, the target nucleic acid is a nucleic acid molecule from an infectious agent selected from a coronaviridae virus, a respiratory syncytial virus (RSV), a polio virus, a West Nile virus, a Chikungunya virus, an Ebola virus, a Lassa virus, a Dengue virus, a SARS coronavirus, a Middle East Respiratory Syndrome (MERS) coronavirus, a Junin virus, a hepatitis C virus, a hepatitis B virus, an Influenza A virus, an Influenza B virus, an Influenza C virus, a vaccinia virus, a variola virus, a polyomavirus, a Pox virus, a Herpes virus, a cytomegalovirus (CMV), a human immunodeficiency virus, a JC virus, a JC polyomavirus (JCV), a BK polyomavirus (BKV), a Simian virus 40 (SV40), a Monkeypox virus, a Marburg virus, a Bunyavirus, an arenavirus, an alphavirus, or a flavivirus. In some embodiments, the target nucleic acid is from a coronaviridae virus selected from SARS-COV-1, SARS-COV-2, MERS-COV, β-coronaviruses, HCoV-OC43, HCoVHKU1, HCoV-NL63, or HCoV-229E.

Also provided is a a nucleic acid amplification process comprising the recombinant nucleic acid primer as described herein. Such amplification process can comprise or consist of an isothermal amplification selected from Loop-mediated isothermal amplification (LAMP or Loopamp); strand displacement amplification (SDA); recombinase polymerase amplification (RPA); helicase dependent amplification (HDA); Isothermal Multiple Displacement Amplification (IMDA); Rolling Circle Amplification (RCA); signal-mediated amplification of RNA technology (SMART); polymerase spiral reaction (PSR); or nicking enzyme amplification reaction (NEAR). In some embodiments, the nucleic acid amplification process can comprise or consist of a recombinase polymerase amplification (RPA); strand displacement amplification (SDA); or helicase dependent amplification (HDA). In some embodiments, the nucleic acid amplification process can comprise or consist of a recombinase polymerase amplification (RPA). In some embodiments, the nucleic acid amplification process can comprise or consist of a a homologous recombination enzyme directed isothermal amplification comprising a recombinase enzyme.

In some embodiments, the nucleic acid amplification process can comprise or consist of a a homologous recombination enzyme directed isothermal amplification. The method comprises or consists of the steps of: (i) contacting a recombinase with the recombinant nucleic acid primer of this disclosure to form an oligo-recombinase complex; (ii) contacting the oligo-recombinase complex to the complementary double stranded target nucleic acid under conditions that allow the oligo-recombinase complex to invade the double stranded target nucleic acid; (iii) admixing a forward primer, a reverse primer, a polymerase, and deoxynucleotide triphosphates (dNTPs) to the process under conditions that allow the forward and reverse primers to bind to their respective complementary regions on the double stranded target nucleic acid, and to allow the polymerase to extend the 3′ end of the forward and reverse primers with dNTPs to generate a first and second amplified double-stranded nucleic acid; and optionally (iv) continuing the amplification process through repetition of (i) and (iii) until a desired degree of amplification product is produced. In some embodiments, the oligo-recombinase complex invasion of the double stranded target nucleic acid denatures the region of the double stranded target nucleic acid that is complementary to the recombinant nucleic acid primer. The steps of the method can be sequential or concurrent.

In some embodiments, the method is performed in the absence of a recombinase loading factor. In some embodiments, the nucleic acid is amplified in the presence of single stranded DNA binding molecule (SSB). Non-limiting examples of SSB include for example an E. coli SSB protein, a gp32 protein from any bacteriophage; a gp32 protein derived from a myoviridae bacteriophage, a T4 gp32 protein, Rb69 gp32 protein, or derivatives of each thereof.

Additional non-limiting examples of recombinase include for example an E. coli RecA, a UvsX from any bacteriophage species, T4 UvsX, Rb69 UvsX, T2 UvsX, T6 UvsX, Ach1 UvsX, KvP40 UvsX, or any derivatives or any combinations of each thereof.

Also provided herein is a composition or amplification system comprising or consisting essentially of (as the active primer element) the recombinant nucleic acid primer as described herein. In some embodiments, the composition comprises or consists essentially (as the active recombination elements) of: (i) the recombinant nucleic acid primer; (ii) a recombinase; (iii) a polymerase (iv) a target nucleic acid; and/or (v) a crowding agent; and/or (vi) a single stranded DNA binding protein (SSB). In some embodiments, the composition or system is free of a recombinase loading factor. In some embodiments, the composition or system further comprises or consists essentially of one or more primer set, wherein each primer set comprises a forward primer and a reverse primer, and each primer set amplifies a complementary target nucleic acid. In some embodiments, the composition is dried or lyophilized.

One aspect of the present disclosure provides an amplification device comprising a composition or system as described herein. In some embodiments, the device is used in a method for determining the presence, absence, or quantity of one or more target nucleic acids in a sample. In some embodiments, the method comprises or consists essentially of, or consist of: (i) amplifying (if present) the one or more target nucleic acids in the sample using the nucleic acid primer and/or system described herein; (ii) directly or indirectly binding the amplified target nucleic acids to one or more signaling agents and detecting the signaling agents if the target nucleic acids are present; (iii) detecting the amplified target amplified nucleic acids if present in the sample. If the target nucleic acids are not present in the sample, no signal is detected.

In another aspect the method comprises, or consists essentially of, or consist of: (i) introducing the sample suspected of comprising the one or more target nucleic acids into a fluid in a a sample preparation reservoir of a sample analysis cartridge; (ii) amplifying the one or more target nucleic acids in the sample if present, using the nucleic acid primer and/or system described herein; (iii) directly or indirectly binding the amplified target nucleic acids to one or more signaling agents and detecting the amplified nucleic acids if present; (iv) wherein in one aspect, detecting the amplified target amplified nucleic acids if present in the sample that comprises releasing the fluid from the sample preparation reservoir into an analysis channel comprising a sensor such that the one or more amplified target nucleic acids directly or indirectly bind to the one or more signaling agents; (v) reacting a substrate with the signaling agent localized over the sensor; (vi) electrically stimulating the reacted substrate-signaling agent complex to generate one or more signals that are detected by the sensor, wherein the signal for each substrate-signaling agent complex is based on the respective potential of the signaling agent for the one or more amplified nucleic acid targets; and (viii) processing the one or more signal to determine the presence, absence, or quantity of the one or amplified target nucleic acid. If the target nucleic acids are not present in the sample, no signal is detected.

In some embodiments, the cartridge comprises the composition as described herein. In some embodiments, the one or more signaling agents each has a detectable potential specific for each of the one or more amplified target nucleic acids. In some embodiments, the detectable potential for each one or more amplified nucleic acids is different.

### BRIEF DESCRIPTION OF THE DRAWINGS

FIG. 1 shows a schematic representation of the structure of an unmodified Invasion Oligonucleotide (IO primer) comprising three sub-regions. Listed from 5′ to 3′, the sub-region are (1) a poly-dC sub-region (length: 7-13 bases), (2) a DNA sub-region that is homologous to the target (length: 25-35 bases), and (3) a methylated RNA sub-region that is homologous to the target (length: 7-13 bases). The methylated RNA region comprises 2′-O-methyl RNA (2′ Ome) nucleotides.

FIG. 2 shows a schematic representation of a non-specific amplification product obtained when the original IO primer was used. In particular, the IO primer was used as a template triggering self-templatization which is the cause of false positive results in COVID-19 PCT test. The COVID19 IO Primer and the internal control reverse primer both unexpectedly aligned to generate some of the hybrid sequence reads observed in the sequencing of products from COVID-19 amplification reactions.

FIG. 3 shows a schematic representation of the structure of an improved and novel recombinant nucleic acid primer design that was optimized for low to no False Positive Rate (FPR) in multiplexed assay. The improvement includes the addition of (1) an extra 2′-O-methyl RNA (2′OME) sites and (2) a 5′ hairpin loop region.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

One aspect of the present disclosure provides a recombinant nucleic acid primer (also referred to herein as an oligonucleotide or probe). Another aspect of the present disclosure provides a nucleic acid amplification process (e.g., isothermal amplication process) comprising a recombinant nucleic acid primer as described herein. Another aspect provides a composition comprising the recombinant nucleic acid primer. Yet another aspect provides an amplification device comprising a composition and/or a recombinant nucleic acid primer (oligonucleotide) as described herein.

It is to be appreciated that certain aspects, modes, embodiments, variations and features of the present methods are described below in various levels of detail in order to provide a substantial understanding of the present technology.

In one aspect, the present disclosure is based on the engineering of a novel recombinant nucleic acid primer for use as a non-functional primer that binds to an enzyme (a recombinase such as UvsX) to facilitate the invasion of double stranded nucleic acids and to unzip the double stranded DNA during isothermal amplification. The novel recombinant nucleic acid primer improves the sensitivity and efficiency of the isothermal amplification process and eliminates the rate of false positive and/or false negative tests, when used in a single or multiplex assays for the diagnosis and prognosis of diseases. Improving the accuracy of multiplex tests, such as the COVID-19 test, is important because a false negative test may result in unnecessary stress and unnecessary weeks-long isolation of individuals.

Also provided herein is a recombinant nucleic acid primer (oligonucleotide; IO primer) comprising or consisting of: (i) a hairpin region; (ii) a first methylated RNA region that is complementary to a sequence of a target nucleic acid; (iii) a target binding region that is complementary to a sequence of the target nucleic acid; and (iv) a second methylated RNA region that is complementary to a sequence of the target nucleic acid. The novel recombinant nucleic acid primer provides a highly specific and sensitive amplification process with reduced non-specific amplification products.

The novel recombinant nucleic acid primer (Invasion oligonucleotide (IO) primer) design is resistant to non-specific amplification. This improves the isothermal amplification chemistry to perform well in a clinical setting using a multiplex assay. In particular, the novel recombinant nucleic acid primer (IO primer) design greatly improves the specificity and sensitivity of multiplexed (2-plex, 3-plex, 4-plex, etc.) assays.

Much of the recombinant nucleic acid primer (i.e. IO primer) contain a large region that is homologous (100% complementary) to the target region of a template nucleic acid. The recombinant nucleic acid primer also comprises an overlapping region with the forward and reverse primers (15-25 nucleotides each) used in the isothermal amplification reaction. The reverse and forward primers are designed to be partly homologous to regions of the IO primer (less than 50% of the forward and reverse primer length). Applicant observed that the forward or reverse primers annealed to the IO primer known in the art it caused the strand displacing polymerase to amplify the recombinant nucleic acid primer beyond the 5′ end resulting in the amplification of the recombinant nucleic acid primer (i.e., self-templating amplification). See FIG. 2. The amplified products of such a reaction were non-specific product caused by self-templating amplification. The self-templating amplification is a particular issue when introducing the IO primer because much of the IO primer and the recombinant nucleic acid primer contain a large region that is homologous to the target region of the template nucleic acid. Therefore, when copies of the IO are amplified via-self templatization, the amplified product comprises nearly the full target region of the template nucleic acid.

The overlapping region with the IO primer and recombinant nucleic acid primer helps contribute to the mechanism of self-templatization, which was found to be the cause of false positive results in multiplex assays, such as the COVID-19 test. In contrast, Applicant's recombinant nucleic acid primer prevents self-templated amplification. In particular, the hairpin structure and the first methylated RNA region of Applicant's recombinant nucleic acid primer ensure that the forward or reverse primer does not anneal to Applicant's primer and ultimately, the strand displacing polymerase does not amplify the recombinant nucleic acid primer beyond the 5′ end of the recombinant nucleic acid primer, which was commonly observed with analogous blocking primer known and used in the art (e.g. IO primer), which lacks this hairpin structure.

In some embodiments, the hairpin region comprises a 5′ hairpin loop region and a stem region. In some embodiments, the 5′ hairpin loop region comprises an adjacent portion to the poly-C region and a non-adjacent portion. The non-adjacent portion is 5′ to the adjacent portion and the non-adjacent portion of the hairpin hybridizes to the stem region, and the non-adjacent portion of the hairpin loop region comprises or consist of a polydeoxyguanylic acid (poly-dG) sequence that is complementary to a polydeoxycytidylic acid (poly-dC) region of the stem region. In some embodiments, the non-adjacent portion of the hairpin loop region comprises a polydeoxycytidylic acid (poly-dC) sequence that is complementary to a polydeoxyguanylic acid (poly-dG) sequence of the stem region. In some embodiments, the stem region comprises a polydeoxycytidylic acid (poly-dC) region; and/or wherein the stem region comprises at least about 7-13 contiguous dC bases.

When the polydeoxycytidylic acid (poly-dC) region of the stem region interacts with the poly-dG of the hairpin loop structure, a natural secondary structure formed from the poly-dGs poly-dCs pairing. Because of the bonds of the C-G stem of the hairpin region are particularly strong, the C-G stem of the hairpin region prevents the strand displacing polymerase from processing through the C-G region. Applicant designed the stem region of the hairpin to slow down this false-positive phenomenon.

In some embodiments, the recombinant nucleic acid primer comprises or consist of a first methylated RNA region that is complementary to a sequence of a target nucleic acid; and a second methylated RNA region that is complementary to a sequence of the target nucleic acid. In some embodiments, the first methylated RNA or the second methylated region comprises 2′-O-methyl RNA (2′OME) nucleotides. In another aspect, the recombinant nucleic acid primer comprises a first methylated RNA very close the 5′ end of the recombinant nucleic acid primer (just 3′ of the hairpin region) and 3′ ends of the the recombinant nucleic acid primer. These 2′ O-methylated RNA help to “knock off” the polymerase. They act as “speed bumps” that help kick off the strand displacing polymerase from the recombinant nucleic acid primer because the polymerase has a hard time processing through the 2′ O-methyl bases. In one aspect, they are proximal to the 5′ end and proximal to the 3′ end to prevent the self-templatization phenomenon by not allowing the strand displacing polymerase to use the recombinant nucleic acid primer as template. Together, the hairpin structure and the first and second methylated RNA force amplification to be more specific because in either direction, there are multiple features (methylated RNA or other modified bases that the polymerase struggles to process through) or the stem loop of the hairpin structure in the 5′ end of the recombinant nucleic acid primer that make it difficult for the IO to be a template for replication, which would lead to false positive results.

Another aspect of the present disclosure provides a nucleic acid amplification process (e.g., isothermal amplication process) comprising a recombinant nucleic acid primer as described herein. Another aspect provides a composition comprising the recombinant nucleic acid primer. Yet another aspect provides an amplification device comprising a composition and/or a recombinant nucleic acid primer (oligonucleotide) as described herein.

One aspect of the present disclosure provides a recombinant nucleic acid primer (oligonucleotide; IO primer) comprising: a hairpin region; and a target binding region that is complementary to a sequence of a target nucleic acid. The target binding region comprises at least one modified nucleoside (base) to prevent amplification of the recombinant nucleic acid primer by a polymerase.

### I. Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art. As used herein with respect to numerical ranges, the terms “approximately,” “about,” “substantially,” and similar terms will be understood by persons of ordinary skill in the art and will vary to some extent depending upon the context in which it is used. If there are uses of the terms that are not clear to persons of ordinary skill in the art, given the context in which it is used, the terms will be plus or minus 10% of the disclosed values. When “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or illustrative language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “sample” or “nucleic acid sample” refers to any substance containing or presumed to contain a nucleic acid, and includes, for example, cellular extracts, tissue extracts, or fluid extracts, or any polynucleotide(s) purified or isolated from such cellular, tissue, or fluid extracts, including, but not limited to, plasma, serum, sputum, skin, spinal fluid, lymph fluid, synovial fluid, urine, tears, blood cells, organs, tumors, whole blood, bone marrow, amniotic fluid, hair, semen, anal secretions, vaginal secretions, perspiration, saliva, buccal swabs, and also to samples of in vitro cell culture constituents (including, but not limited to, conditioned medium resulting from the growth of cells (including prokaryotic and eukaryotic cells) in cell culture medium, recombinant cells, and cell components).

Samples can comprise cellular or tissue explants obtained from an individual or organism during a medical procedure or intervention, such as a surgical procedure or biopsy. Nucleic acid samples from environmental sources are also included among “samples” to which the methods described herein can be applied. It will be appreciated that target polynucleotides can be isolated from such samples using any of a variety of procedures known in the art. It will be appreciated that target polynucleotides can be cut or sheared prior to analysis, including the use of such procedures as mechanical force, sonication, restriction endonuclease cleavage, or any method known in the art. In general, the target polynucleotides of the present teachings will be single stranded, though in some embodiments the target polynucleotide can be double stranded, and a single strand can result from denaturation. In some embodiments of the methods described herein, there is no separate isolation step, and the methods are directly performed on a sample of interest, such as, for example, cellular extracts or lysates, tissue extracts or lysates, or fluid extracts.

As used herein, the term “nucleic acid,” “polynucleotide,” or “oligonucleotide” generally refers to any polyribonucleotide or poly-deoxyribonucleotide, and includes unmodified RNA, unmodified DNA, modified RNA, and modified DNA. Polynucleotides include, without limitation, single- and double-stranded DNA and RNA polynucleotides. The term polynucleotide, as it is used herein, embraces chemically, enzymatically or metabolically modified forms of polynucleotides, as well as the naturally occurring chemical forms of DNA and RNA found in or characteristic of viruses and cells, including for example, simple (prokaryotic) and complex (eukaryotic) cells. A nucleic acid polynucleotide or oligonucleotide as described herein retains the ability to hybridize to its cognate complimentary strand. A nucleic acid sample will comprise nucleic acids that serve as templates for and/or substrates for a polymerization reaction. A polynucleotide useful for the methods described herein can be an isolated or purified polynucleotide; it can be an amplified polynucleotide in an amplification reaction, or a transcribed product from an in vitro transcription reaction.

Accordingly, as used herein, the term nucleic acid, polynucleotide or oligonucleotide also encompasses primers and probes, as well as oligonucleotide fragments, and is generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases (including, but not limited to, abasic sites). There is no intended distinction in length between the term “nucleic acid,” “polynucleotide,” and “oligonucleotide,” and these terms are used interchangeably. These terms refer only to the primary structure of the molecule. An oligonucleotide is not necessarily physically derived from any existing or natural sequence, but can be generated in any manner, including chemical synthesis, DNA replication, DNA amplification, reverse transcription or any combination thereof.

As used herein, the term “nonextendable nucleotide” refers to nucleotides that prevent extension of a polynucleotide chain by a polymerase. Examples of such nucleotides include dideoxy nucleotides (ddA, ddT, ddG, ddC) that lack a 3′-hydroxyl on the ribose ring, thereby preventing 3′ extension by DNA polymerases. Other examples of such nucleotides include, but are not limited to, inverted bases, which can be incorporated at the 3′-end of an oligo, leading to a 3′-3′ linkage, which inhibits extension by DNA polymerases. Additional nonextendable nucleosides are discussed herein.

As used herein, the terms “target nucleic acid,” “target RNA,” “target DNA,” “target oligonucleotide,” and “target polynucleotide,” refer to a nucleic acid of interest, e.g., a nucleic acid of a particular nucleotide sequence one wishes to amplify, detect and/or quantify in a sample using the approaches described herein. The target polynucleotide can be obtained from any source, and can comprise any number of different compositional components. For example, the target can be nucleic acid (e.g. DNA or RNA), transfer RNA, sRNA, and can comprise nucleic acid analogs or other nucleic acid mimic. The target can be methylated, non-methylated, or both. The target can be bisulfate-treated and non-methylated cytosines converted to uracil. Further, it will be appreciated that “target polynucleotide” can refer to the target polynucleotide itself, as well as surrogates thereof, for example amplification products, and native sequences. In some embodiments, the target polynucleotide is a nucleic acid sequence comprising a rare mutation. The terms can refer to a single-stranded or double-stranded polynucleotide molecule (e.g., RNA, DNA, as the case may be), or a specific strand thereof, to which, for example, an oligonucleotide primer that is “specific for” the target nucleic acid anneals or hybridizes. A target nucleic acid as used herein has at least a portion of sequence that is complementary (e.g., 39-61 bases in length) to a target-specific oligonucleotide molecule, such as hairpin barcode primer.

As used herein, the term “False Positive Rate” or “FPR” refers to the quotient of the number of negative samples reported as positive (false positive) divided by the sum of the total number of negative samples of the false positive and true negative (FPR=[False Positive]/(False Positive+True Negative)]). In some embodiments, the FPR means the quotient of the total number of false positives (FP) over the sum of the total number of false positives (FP) and the total number of true negatives (TN) (FPR=FP/(FP+TN)).

As used herein, true positive rate (TPR) is the quotient of the total number of true positives (TP) over the sum of the total number of true positives (TP) and the total number of false negatives (FN) (PR=TP/(TP+FN)). As used herein, the term “true negative rate” or “TNR” means the quotient of the total number of true negatives (TN) over the sum of the total number of false positives (FP) and (TN) (TNR=TN/(FP+TN)). As used herein, “false negative rate” or “FNR” is the quotient of the total number of false negatives (FN) over the sum of the total number of true positives (TP) and the total number of false negatives (FN) (FNR=FN/(TP+FN)).

As used herein, the term “hairpin” or “stem-loop” refers to the partially double-stranded region or structure of the stem-loop primer that forms when the primer is in the closed configuration.

As used herein, the terms “complementary” or “complementarity” are used in reference to polynucleotides (i.e., a sequence of nucleotides such as an oligonucleotide or a target nucleic acid) related by the base-pairing rules. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tm of the formed hybrid. “Hybridization” methods involve the annealing of one nucleic acid to another, complementary nucleic acid, i.e., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modern biology.

The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present disclosure and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

As used herein, an “oligonucleotide primer” refers to a polynucleotide molecule (i.e., DNA, RNA, artificial nucleotides or a combination thereof) capable of annealing to a portion of a sequence of a target nucleic acid, and providing a 3′ end substrate for a polymerase enzyme to produce an enzymatic extension product that is complementary to the nucleic acid to which the polynucleotide is annealed. An oligonucleotide primer can refer to more than one primer and can be naturally occurring, as in, for example, a purified restriction digest, or can refer to a molecule produced synthetically. An oligonucleotide primer can act as a point of initiation for the synthesis of a strand complementary to a sequence of a target nucleic acid, when placed under conditions in which primer extension can be catalyzed. A primer is preferably single-stranded for maximum efficiency in amplification. The conditions for initiation and extension usually include the presence of four different deoxyribonucleoside triphosphates (dNTPs) and a polymerization-inducing agent, such as a DNA polymerase or a reverse transcriptase, in a suitable buffer (“buffer” includes constituents that are cofactors for the enzymatic reactions, and/or which affect pH, ionic strength, etc.) and at a suitable temperature.

As used herein, the term “Multiplex amplification” refers to amplification of multiple different target nucleic acid sequences in the same reaction (see, e.g., PCR PRIMER, A LABORATORY MANUAL (Dieffenbach, ed. 1995) Cold Spring Harbor Press, pages 157-171). “Multiplex amplification,” as used herein, refers to amplification of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30 or more targets, e.g., at least 50, at least 100, at least 250, at least 500, at least 750, at least 1000, at least 5000 or more, targets.

As used herein, the term “comprising” or “comprises” is used in reference to compositions, methods, and respective component(s) thereof, that are useful to an embodiment, yet open to the inclusion of unspecified elements, whether useful or not.

As used herein, the term “consisting essentially of” refers to those elements for a given embodiment. The term permits the presence of elements that do not materially affect the basic and novel or functional characteristic(s) of that embodiment of the disclosure.

As used herein, the term “Invasion oligonucleotide” or “Invasion primer” or “IO primer” refers to an oligonucleotide that hybridizes to a target nucleic acid at a location near the region of hybridization between primer binding sites on the target nucleic acid, wherein the Invasion primer comprises a portion (e.g., a chemical moiety, or nucleotide-whether complementary to that target or not) that overlaps with the region of hybridization between the forward and reverse primer binding sites on the target. See e.g., Hoser et al., PlosOne 9 (11): e112656 (2014) DOI: 10.1371/journal.pone.0112656.

### II. Recombinant Nucleic Acid Primer

In one aspect, the present disclosure provides a recombinant nucleic acid primer (i.e., modified invasion oligonucleotide; hairpin invasion oligonucleotide) comprising: a hairpin region; and a target binding region that is complementary to a sequence of a target nucleic acid. The target nucleic acid can be double stranded or single stranded, and can be DNA or RNA.

In one aspect, the present disclosure provides a recombinant nucleic acid primer (i.e., modified invasion oligonucleotide; hairpin invasion oligonucleotide) comprising or consisting of: (i) a hairpin region; (ii) a first methylated RNA region that is complementary to a sequence of a target nucleic acid; (iii) a target binding region that is complementary to a sequence of the target nucleic acid; and (iv) a second methylated RNA region that is complementary to a sequence of the target nucleic acid. As shown in FIG. 3, the recombinant nucleic primer is about 39-100 bases in length and contains four sub-regions. The sub-regions, listed from 5′ to 3′ are: (1) a hairpin loop structure that is about 10-20 bases; (2) a poly-dC sub-region (hairpin stem structure) with about 7-13 bases in length; (3) a DNA sub-region that was homologous to the target nucleic acid region and was about 25-35 bases or about 25-50 bases in length; and (4) a first methylated region that is I about 1-4 nucleotides; and (5) a methylated RNA sub-region that is length: 7-13 bases.

During homologous recombination enzyme directed isothermal amplification process of the present disclosure, a recombinase binds to and coats a single-stranded DNA (ssDNA; primer, or probe) to form a nucleoptorein complex or filament. This nucleoprotein complex or filament then scans the target double-stranded DNA (dsDNA) molecule to identify the regions of sequence homology with the bound ssDNA (primer or probe). The location of a homologous duplex by the recombinase nucleoprotein/filament results in the invasion (opening) of the target double stranded DNA, and the formation of a homologous joint between the ssDNA and the double stranded DNA. This in turn leads to strand exchange, during which, the ssDNA contained within the nucleoprotein filament forms Watson-Crick base pairs with the complementary strand of the “target” double stranded DNA, displacing the noncomplementary strand in the dsDNA. The hybrid DNA formed by strand exchange is further processed by a polymerase and other recombination factors, eventually yielding to two intact DNA duplexes.

During isothermal amplification known in the art, such as the RPA, the forward and/or reverse primer serves as the single stranded DNA that binds to a recombinase to facilitate invention of the double stranded DNA. In the present disclosure, a recombinant nucleic acid primer (also referred to as a long/non-extendable oligonucleotide, a modified invasion oligonucleotide primer; modified IO primer; oligonucleotide) functions as a nonfunctional primer whose sole role is to bind the recombinase to facilitate the invasion of the target double stranded DNA and unwind the dsDNA. The recombinant nucleic acid primer of the present disclosure facilitates the separation of double stranded nucleic acid duplex during an isothermal amplification, thereby allowing the forward and reverse primers to bind to their respective complementary single stranded DNA on the target molecule independent of a recombinase enzyme. However, the recombinant nucleic acid primer does not compete with primers for the target nucleic acid binding during an amplification reaction. The structure of the recombinant nucleic acid primer facilitates its function during the isothermal amplification process.

### 1. Hairpin Region

In some embodiments of the recombinant nucleic acid primer, the hairpin region comprises a 5′ hairpin loop region and a stem region. A hairpin region on the 5 ‘end of the recombinant nucleic acid was designed to create a rigid stem that prevents the recombinant nucleic acid primer from acting as its own template. In some embodiments, the 5’ hairpin loop region is about 25 to about 65 nucleotides in length.

In some embodiments, the the 5′ hairpin loop region comprises an adjacent portion to the stem region and a non-adjacent portion. In some embodiments, the non-adjacent portion is 5′ to the adjacent portion and the non-adjacent portion of the hairpin loop region hybridizes to the stem region.

In some embodiments, the 5′ terminal portion of the 5′ hairpin loop region is between 10 and 30 bases in length (e.g., 10, 11, 12, 13, 14, 15, 16, 17, 18, 19, 20, 21, 22, 23, 24, 25, 26, 27, 28, 29, or 30 bases in length). In other embodiments, the 5′ terminal portion of the 5′ hairpin loop region is between 15 and 25 bases in length. In some embodiments, the 5′ hairpin loop region of the recombinant nucleic acid primer is between 8 and 15 nucleotides in length (e.g., 8, 9, 10, 11, 12, 13, 14, or 15 nucleotides).

The hairpin region typically includes from about 18 to about 50 nucleotides, more typically from about 25 to about 40 nucleotides. In some embodiments, the non-adjacent portion of the hairpin loop region comprises a polydeoxyguanylic acid (poly-dG) sequence that is complementary to a polydeoxycytidylic acid (poly-dC) region. In some embodiments, the poly-dC region is part of the stem region. In some embodiments, the non-adjacent portion of the hairpin loop region comprises a polydeoxycytidylic acid (poly-dC) sequence that is complementary to a polydeoxyguanylic acid (poly-dG) region of the stem region.

Formation of the hairpin structure is accomplished via design of two subregions having substantial anti-parallel complementarity, i.e., sufficient complementarity running in opposite directions to specifically hybridize under stringent conditions, thereby forming a stem region. Between the substantially mutually complementary regions is interposed a non-complementary sequence, which does not hybridize intramolecularly and therefore has as a single-stranded loop configuration upon formation of the double-stranded stem region. The number and base composition of nucleotides in the loop region are selected to allow the mutually complementary subregions of the stem to hybridize. For example, the loop region is designed to avoid substantial complementarity with either strand of the stem region and typically has from 3 to 20 nucleotides, more typically from 3 to 10 nucleotides, and most typically from 5 to 8 nucleotides.

In some embodiments, the number and base composition of nucleotides in the stem region are selected for substantial complementarity of two regions running in opposite directions such that a double-stranded hybrid of a desired relative stability is formed under stringent hybridization conditions. Typically, the stem region typically has from 14 to 46 nucleotides (i.e., 7-23 nucleotides for each strand of the stem), more typically from 16 to 40 nucleotides (i.e., 8-20 nucleotides for each strand of the stem), and most typically from 20 to 32 nucleotides (i.e., 10-16 nucleotides for each strand of the stem). The target-binding region is non-overlapping with the hairpin region and is typically at least 3 or at least 6 nucleotides, more typically at least 10 nucleotides, even more typically at least at least 14 or at least 17 nucleotides, and still more typically at least 25 or at least 30 nucleotides in length. Target-binding regions having complementary sequences over stretches greater than 20 bases in length are generally preferred.

The length and base composition of the target-binding region is generally selected to not interfere with formation of the attached hairpin structure and to not in itself form a stem-loop structure with equal or greater thermal stability that the labeled stem-loop structure under hybridization conditions appropriate for specific detection of the target nucleic acid. The nucleotide sequence of the target-binding region can be, e.g., predetermined, random, or degenerate. For example, the predetermined target binding region can be designed to have substantial complementarity with a predetermined polynucleotide such as, e.g., a nucleic acid associated with an infectious agent (e.g., viral nucleic acids such as, for example, HIN or EBV nucleic acids).

In some embodiments, the the target nucleic acid is a nucleic acid molecule from an infectious agent selected from a coronaviridae virus, a respiratory syncytial virus (RSV), a polio virus, a West Nile virus, a Chikungunya virus, an Ebola virus, a Lassa virus, a Dengue virus, a SARS coronavirus, a Middle East Respiratory Syndrome (MERS) coronavirus, a Junin virus, a hepatitis C virus, a hepatitis B virus, an Influenza A virus, an Influenza B virus, an Influenza C virus, a vaccinia virus, a variola virus, a polyomavirus, a Pox virus, a Herpes virus, a cytomegalovirus (CMV), a human immunodeficiency virus, a JC virus, a JC polyomavirus (JCV), a BK polyomavirus (BKV), a Simian virus 40 (SV40), a Monkeypox virus, a Marburg virus, a Bunyavirus, an arenavirus, an alphavirus, or a flavivirus. In some embodiments, the target nucleic acid is from a coronaviridae virus selected from SARS-COV-1, SARS-COV-2, MERS-COV, β-coronaviruses, HCoV-OC43, HCoVHKU1, HCoV-NL63, or HCoV-229E.

In some embodiments, the stem region of the hairpin region comprises a polydeoxycytidylic acid (poly-dC) region. In some embodiments, the stem region comprises at least about 1-20; at least about 5-20; at least about, 6-20; at least about 7-20; at least about 10-20; at least about 1-15, at least about 5-15, at least about about 6-15, at least about 7-15; at least about 10-15; at least about 1-13; at least about 5-13; at least about 6-13; at least about 7-13; at least about 10-13; at least about 1-10; at least about 5-10; at least about 6-10 contiguous dC bases. In some embodiments, the stem region comprises at least about 7-13 contiguous dC bases.

### 2. Modified Nucleosides

The recombinant nucleic acid primer of the present disclosure facilitates the separation of double stranded nucleic acid duplex during an isothermal amplification, thereby allowing the forward and reverse primers to bind to their respective complementary single stranded DNA on the target molecule independent of a recombinase enzyme. However, the recombinant nucleic acid primer does not compete with primers for the target nucleic acid binding during an amplification. In addition, the recombinant nucleic acid primer of the present disclosure is not a substrate for a DNA polymerase because the target binding region comprises at least one modified nucleoside (base) to prevent amplification of the recombinant nucleic acid primer by a polymerase. A number of nucleoside modifications that block polymerase amplification of an oligonucleotide are known to the skilled artisan. There are over 200 commercially available DNA and/or RNA oligonucleotides modifications to the skilled artisan. These modified nucleoside can be found for example at Sigma-Aldrich or Integrated DNA Technologies. See e.g., sigmaaldrich.com/deepweb/assets/sigmaaldrich/marketing/global/documents/373/354/custom-oligonucleotide-modifications-guide.pdf; idtdna.com/site/catalog/modifications/category/7. In some embodiments, the target binding region of the recombinant nucleic acid primer (oligonucleotide) comprises at least one modified nucleoside (base) to prevent amplification of the recombinant nucleic acid primer by a polymerase.

In some embodiments, the at least one modified nucleoside (base) is selected from 5-nitroindole; 8-oxo-2′-deoxyguanosine (8-oxo-dG); 8-oxo7,8-dihydro-2′-deoxyguanosine; 8-oxo-deoxyadenosine (8-oxo-dA); 5-hydroxymethyl deoxycytosine (5-hydroxymethyl-dC); 5′-dimethoxytrityl-N-benzoyl-3′-deoxyadenosine,2′-succinoyl-long chain alkylamino-CPG (3′-dA-CPG); 5′-dimethoxytrityl-N-dimethylformamidine-3′-deoxyguanosine,2′-succinoyl-long chain alkylamino-CPG (3′-dG-CPG); 5′-dimethoxytrityl-N-benzoyl-3′-deoxyCytosine,2′-succinoyl-long chain alkylamino-CPG (3′-dC-CPG); 5′-dimethoxytrityl-3′-deoxythymidine,2′-succinoyl-long chain alkylamino-CPG (3′-dT-CPG); 3′-deoxyadenosine; cytosine arabinoside; inverted dT; 2′-O-methyl RNA nucleotide; 2′-O-methyladenosine (2′-OMe-A); 2′-O-methylcytosine (2′-OMe-C); 2′-O-methylguanosine (2′-OMe-G); 2′-O-methyluridine (2′-OMe-U); inosine, or a modified base that blocks 5′ to 3′ polymerase amplification. In some embodiments, the modified nucleoside is 5-Nitroindole directs random incorporation of any specific base when used as a template for DNA polymerase and partially blocks the polymerase processivity. In some embodiments, the modified nucleoside is Dideoxycytidine (ddC), which is a 3′ chain terminator that prevents 3′ extension by DNA polymerases. In some embodiments, the modified nucleoside is an Inverted dT. Inverted dT are incorporated at the 3′-end of an oligo to inhibit extension by DNA polymerases. In addition Inverted dT inhibits the degradation by 3′ exonucleases. In some embodiments, the modified base is an oxo base such as 8-oxo-dA or 8-oxo-dG. Oxo bases can be placed at the 5′ end or within an internal region of the recombinant nucleic acid primer to inhibit DNA polymerization. 3′-dA-CPG, -dC-CPG, -dG-CPG, or -dT-CPG can be placed at the 3′ end of the recombinant recombinant nucleic acid primer to inhibit extension by DNA polymerases. Ara-C is a chemotherapeutic agent that inhibits DNA replication and can be placed within the target binding region of the recombinant nucleic acid primer.

In some embodiments, at least one modified nucleoside is a di-deoxy-nucleotide selected from dideoxycytidine (ddC); 2′-3′-dideoxycytidine (2′,3′ dideoxy-C); 5′-Dimethoxytrityl-N-succinoyl-long chain alkylamino-CPG, 2′,3′-deoxycytosine (2′,3′-ddC-CPG); 2′-3′-dideoxyadenosine (2′,3′ dideoxy-A); or 5′-dimethoxytrityl-N-succinoyl-long chain alkylamino-CPG, 2′,3′-deoxyadnosinc (2′,3′-ddA-CPG).

In some embodiments, the target binding region comprises a base modified with a C3 spacer amidite (DMT-1,3-propanediol); a C3 spacer phosphoramidite; a C3 spacer; a C6 spacer; hexanediol; a triethylene glycol spacer (spacer 9); a 18-atom hexa-ethyleneglycol spacer (spacer 18); 1′,2′-Dideoxyribose (dSpacer); a C6 amine; a peptide nucleic acid (PNA); a locked nucleic acid (LNA); or a 1-Dimethoxytrityloxy-propanediol-3-succinoyl)-long chain alkylamino-CPG (3′-Spacer-C3-CPG).

In one embodiment, at least one modified base that inhibits amplification by the polymerase is located at one or more of a 3′-end; a 5′-end; or anywhere within the internal region of the target binding region of the recombinant nucleic acid primer to inhibit amplification by the polymerase. In one embodiment, the 3′-end and the 5′-end of the target binding region of the recombinant nucleic acid primer comprise one or more modified bases. In one embodiment, the one or more modified bases are the same or different. In another embodiment, the 3′-end and the internal region of the target binding region comprise the one or more modified bases that are the same or different. In another embodiment, the 5′-end and the internal region of the target binding region of the recombinant nucleic acid primer comprise one or more modified bases. In one embodiment, the one or more modified bases are the same or different. In another embodiment, the 3′-end, the 5′-end, and the internal region of the target binding region of the recombinant nucleic acid primer comprise one or more modified bases. In one embodiment, at least two of the 3′-end modification, the 5′-end modification, and the internal modification are the same or different.

In some embodiments, the modified base is a 2′-O-Methyl RNA (2′OME) nucleotide; optionally, a region comprising at least one 2′OME nucleotide is a methylated RNA region. In some embodiments, the target binding region further comprises a first methylated RNA region and a second methylated RNA region. In some embodiments, the first methylated RNA region comprises at least one 2′OME nucleotide, or at least two 2′OME nucleotides, or at least three 2′OME nucleotides, or at least four 2′OME nucleotides, or at least about five 2′OME nucleotides. In another embodiment, the first methylated RNA region is located at the 5′-end of the target binding region of the recombinant nucleic acid primer. In another embodiment, the second methylated RNA region is located at the 3′-end of the target binding region. In another embodiments, the second methylated RNA region comprises at least 7 to at least 13 contiguous nucleotides. In one embodiment, all the complementary nucleic acid residues in the target binding region comprise 2′OME nucleotides.

### 3. Methylated RNA Regions

In some embodiments, the recombinant nucleic acid comprises two methylated regions. In some embodiments, the first methylated RNA or the second methylated region comprises 2′-O-methyl RNA (2′OME) nucleotides. In some embodiments, the first methylated RNA region is complementary to a sequence of a target nucleic acid. In some embodiments, the first methylated RNA region comprises at least about one 2′OME nucleotide, or at least about two 2′OME nucleotides, or at least about three 2′OME nucleotides, or at least about four 2′OME nucleotides, or at least about about five 2′OME nucleotides. In some embodiments, the first methylated RNA region comprises between about 1-3 2′OME nucleotides, between about 1-6 2′OME nucleotides, between about 1-8 2′OME nucleotides, between about 1-10 2′OME nucleotides, or between about 1-16 2′OME nucleotides. In some embodiments, the first methylated RNA region is located at the 5′-end of the recombinant nucleic acid primer in close proximity to the stem region of the hairpin region.

In some embodiments, the second methylated RNA region is also complementary to a sequence of the target nucleic acid. In some embodiments, the second methylated RNA region is located at the 3′-end of the recombinant nucleic acid primer. In some embodiments, the second methylated RNA region comprises at least about 7 to at least about 13 contiguous nucleotides. In some embodiments, the second methylated RNA region comprises between about 1-7 2′OME nucleotides, between about 1-8 2′OME nucleotides, between about 1-10 2′OME nucleotides, between about 1-12 2′OME nucleotides, between about 1-13 2′OME nucleotides, between about 1-14 2′OME nucleotides, between about 1-21 2′OME nucleotides, between about 1-27 2′OME nucleotides, or between about 1-32 2′OME nucleotides. In some embodiments, all the complementary nucleic acid residues in the first or second methylated RNA region are replaced with 2′OME nucleotides.

2′-O-methyl RNA nucleotides ensure that the recombinant nucleic acid primer is not a viable polymerase substrate or template. DNA primers comprising 2′-O-methyl RNA nucleotides can prime target DNA as efficiently as DNA primers, but they are unable to yield exponential product amplification. As such, the 2′-O-methyl RNA nucleotides act as speed bumps that help kick off the strand displacing polymerase because the polymerase has a hard time processing through the 2′ O-methyl bases.

In some embodiments, the location of the first and second methylated RNA region affect the effectiveness of the recombinant nucleic acid primer. The first and second 2′ O-methylated RNA help to “knock off” the polymerase, thereby preventing the amplification of the recombinant nucleic acid primer. The first and second 2′ O-methylated RNA act as speed bumps that help kick off the strand displacing polymerase because the polymerase has a hard time processing through the 2′ O-methyl bases. The placement of the first and second methylated RNA at the proximal 5′ end and proximal to the 3′ end of the recombinant nucleic acid primer was a strategic decision because at these positions, they help prevent the self-templatization phenomenon observed with IO primers known in the art. Accordingly, the location of the first and second methylated RNA at the proximal 5′ end and proximal to the 3′ end of the recombinant nucleic acid primer also ensure that the polymerase does not use the recombinant nucleic acid primer as template. This forces the isothermal amplification to be more specific because in either direction, there are multiple features (methylated RNA or other modified bases that the polymerase struggles to process through) or the meaty stem loop of the hairpin structure in the 5′ end of the recombinant nucleic acid primer.

The first and second methylated RNA region acting as speed bumps to knock off a polymerase because to avoid the non-specific amplification of the recombinant nucleic acid primer and the high rate of false positive results (FPR), it is critical that the recombinant nucleic acid primer not act as a template. This is critical because the recombinant nucleic acid primer does have near full homology with the target sequence to be amplified, and both the forward and reverse primers have natural overlap with the recombinant nucleic acid primer. In the absence of the first and second methylated RNA regions, isothermal amplification reaction usually create a very real-looking amplicon amplification product because the polymerase isn't stopped in absence of real target region. As such, this design is novel and inventive over the design known in the art.

### 4. Target Binding RNA Region

In some embodiments, the target binding region is complementary to a sequence of the target nucleic acid. In some embodiments, the recombinant nucleic acid primer is not extended by a polymerase, thereby rendering the recombinant nucleic acid primer resistant to amplification by a polymerase.

In some embodiments, the recombinant nucleic acid primer does not comprise a 3′-end inverted nucleotide base modification. In some embodiments, the recombinant nucleic acid primer is resistant to amplification by a polymerase and does not comprise a 3′-end inverted nucleotide base modification. Applicant observed by the inclusion of the 3′-end inverted nucleotide base modification alone was not sufficient to prevent the self-self-templatization phenomenon. In some embodiments, the recombinant nucleic acid primer is not a template for amplification, thereby resistant to non-specific amplification caused by the activity of a forward and/or reverse primers binding.

In one aspect of the present disclosure, the recombinant nucleic acid primer is used in single or a multiplexed assay. When used in a multiplexed assay, a plurality of primers are combined each with a specificity to a distinct target nucleic acid region. In some embodiments, the presence of the recombinant nucleic acid primer significantly enhances the specificity and sensitivity of the multiplexed assay when compare to a multiplexed assay conducted in the absence of the recombinant nucleic acid primer. In some embodiments, the presence of the first and second methylated RNA regions on the recombinant nucleic acid primer as described herein enhances the specificity and sensitivity of a multiplexed assay when compared to a recombinant nucleic acid primer containing only the first or the second methylated RNA region.

In some embodiments, the combination of the 5′ hairpin structure and the first methylated RNA region significantly enhances the specificity and sensitivity of a multiplexed assay when compared to a recombinant nucleic acid primer comprising only the 5′ hairpin structure, the first methylated RNA region, or the second methylated RNA region. In some embodiments, the specificity and sensitivity of multiplexed assay is based on the occurrence of false positive results.

In some embodiments, the recombinant nucleic acid primer comprises one or more additional nucleoside modification. In one embodiment, the one or more additional modification is selected from 5-nitroindole; 8-oxo-2′-deoxyguanosine (8-oxo-dG); 8-oxo7,8-dihydro-2′-deoxyguanosine; 8-oxo-deoxyadenosine (8-oxo-dA); 5-hydroxymethyl deoxycytosine (5-hydroxymethyl-dC); 5′-dimethoxytrityl-N-benzoyl-3′-deoxyadenosine,2′-succinoyl-long chain alkylamino-CPG (3′-dA-CPG); 5′-dimethoxytrityl-N-dimethylformamidine-3′-deoxyguanosine,2′-succinoyl-long chain alkylamino-CPG (3′-dG-CPG); 5′-dimethoxytrityl-N-benzoyl-3′-deoxyCytosine,2′-succinoyl-long chain alkylamino-CPG (3′-dC-CPG); 5′-dimethoxytrityl-3′-deoxythymidine,2′-succinoyl-long chain alkylamino-CPG (3′-dT-CPG); 3′-deoxyadenosine; cytosine arabinoside; inverted dT; inosine, or a modified base that blocks 5′ to 3′ polymerase amplification. In one embodiment, the one or more additional nucleoside modification is a di-deoxy-nucleotide selected from dideoxycytidine (ddC); 2′-3′-dideoxycytidine (2′,3′ dideoxy-C); 5′-Dimethoxytrityl-N-succinoyl-long chain alkylamino-CPG, 2′,3′-deoxycytosine (2′,3′-ddC-CPG); 2′-3′-dideoxyadenosine (2′,3′ dideoxy-A); or 5′-dimethoxytrityl-N-succinoyl-long chain alkylamino-CPG, 2′,3′-deoxyadnosine (2′,3′-ddA-CPG). In another embodiment, the one or more additional nucleoside modification is a nucleoside modified with a C3 spacer amidite (DMT-1,3-propanediol); a C3 spacer phosphoramidite; a C3 spacer; a C6 spacer; hexanediol; a triethylene glycol spacer (spacer 9); a 18-atom hexa-ethyleneglycol spacer (spacer 18); 1′,2′-Dideoxyribose (dSpacer); a C6 amine; a peptide nucleic acid (PNA); a locked nucleic acid (LNA); or a 1-Dimethoxytrityloxy-propanediol-3-succinoyl)-long chain alkylamino-CPG (3′-Spacer-C3-CPG). In some embodiment, the combination of the 2′-O-methyl RNA nucleotide (2′OMe) modifications and the one or more additional modifications significantly improves the sensitivity of the multiplex assay. In some embodiments, the improvement is shown by the enhanced suppression of the auto- and/or self-templatization of the recombinant nucleic acid primer when compared to a recombinant nucleic acid having one type of modification.

Another aspect of the present disclosure provides a recombinant nucleic acid primer comprising: (i) a 5′ hairpin region which comprises a 5′ hairpin loop region that is about 25 to about 65 nucleotides in length; and stem region comprising a polydeoxycytidylic acid (poly-dC) region and/or having at least about 7-13 contiguous dC bases; (ii) a first methylated RNA region that is complementary to a sequence of a target nucleic acid comprising at least about one to at least about five 2′-methyl RNA (2′OME) nucleotides; (iii) a target binding region that is complementary to a sequence of the target nucleic acid, and (iv) a second methylated RNA region that is complementary to a sequence of the target nucleic acid comprising at least about seven to at least about thirteen 2′OME nucleotides. In one embodiment, the recombinant nucleic acid primer is not a template for amplification. In one embodiment, the recombinant nucleic acid primer significantly enhances the specificity and sensitivity of a multiplexed assay when compared to a recombinant nucleic acid primer comprising only the 5′ hairpin region, the first methylated RNA region, or the second methylated RNA region. In one embodiments, the specificity and sensitivity of multiplexed assay is based on the occurrence of false positive results.

In some embodiments, the target nucleic acid is a nucleic acid molecule from an infectious agent selected from a coronaviridae virus, a respiratory syncytial virus (RSV), a polio virus, a West Nile virus, a Chikungunya virus, an Ebola virus, a Lassa virus, a Dengue virus, a SARS coronavirus, a Middle East Respiratory Syndrome (MERS) coronavirus, a Junin virus, a hepatitis C virus, a hepatitis B virus, an Influenza A virus, an Influenza B virus, an Influenza C virus, a vaccinia virus, a variola virus, a polyomavirus, a Pox virus, a Herpes virus, a cytomegalovirus (CMV), a human immunodeficiency virus, a JC virus, a JC polyomavirus (JCV), a BK polyomavirus (BKV), a Simian virus 40 (SV40), a Monkeypox virus, a Marburg virus, a Bunyavirus, an arenavirus, an alphavirus, or a flavivirus. In some embodiments, the target nucleic acid is a nucleic acid molecule from an influenza virus, a hepatitis B virus; an Ebola virus a coronaviridae virus, a SARS coronavirus, a Middle East Respiratory Syndrome (MERS) coronavirus. In some embodiments, target nucleic acid may be a RNA for the detection and/or diagnosis of an influenza or HIV infection, and DNA for DNA viruses or bacteria or RNA for the detection of 16s rRNA for bacteria.

In some embodiments, the target nucleic acid is from a coronaviridae virus selected from SARS-COV-1, SARS-COV-2, MERS-COV, β-coronaviruses, HCoV-OC43, HCoVHKU1, HCoV-NL63, or HCoV-229E.

### 5. Multiplexed Assay

In one aspect of the present disclosure, the recombinant nucleic acid primer is used in a multiplexed assay. As used herein, a multiplexed assay is an amplification assay that that detects multiple distinct target sequences or alleles per reaction. The multiplex assay is used in a device as disclosed herein and/or a device disclosed in for example, U.S. Pat. No. 9,207,245, or U.S. Patent Publication No. US 2020/0408750. In some embodiments, the presence of the recombinant nucleic acid primer significantly enhances the specificity and sensitivity of the multiplexed assay when compare to a multiplexed assay conducted in the absence of the recombinant nucleic acid primer. In some embodiments, the presence of the first and second methylated RNA regions on the recombinant nucleic acid primer as described herein enhances the specificity and sensitivity of a multiplexed assay when compared to a recombinant nucleic acid primer containing only the first or the second methylated RNA region.

Since its introduction in 1988 (Chamberlain, et al. Nucleic Acids Res., 16:11141 (1988)), multiplex PCR has become a routine means of amplifying multiple genetic loci in a single reaction. This approach has found utility in a number of research, as well as clinical, applications. Multiplex PCR has been described for use in diagnostic virology (Elnifro, et al. Clinical Microbiology Reviews, 13:559 (2000)), paternity testing (Hidding and Schmitt, Forensic Sci. Int., 113:47 (2000); Bauer et al., Int. J. Legal Med. 116:39 (2002)), preimplantation genetic diagnosis (Ouhibi, et al., Curr Womens Health Rep. 1:138 (2001)), microbial analysis in environmental and food samples (Rudi et al., Int J Food Microbiology, 78:171 (2002)), and veterinary medicine (Zarlenga and Higgins, Vet Parasitol. 101:215 (2001)), among others. Most recently, expansion of genetic analysis to whole genome levels, particularly for single nucleotide polymorphisms, or SNPs, has created a need for highly multiplexed PCR capabilities. In some embodiments, a multiplexed assay is an assay method that can detect and characterize multiple specific target nucleic acids and/or sequence variations in a single reaction. In some embodiments, the methods of the present disclosure detects single nucleotide polymorphism (SNP) in a sample. In some embodiments, the methods of the present disclosure detects SNPs in one or more genetic loci.

The present disclosure provides methods for substantial multiplexing of isothermal amplification reactions by combining the recombinant nucleic acid primer of the present disclosure with multiplex isothermal amplification. In some embodiments, the recombinant nucleic acid primer enhances the detection step and signal amplification that allows very large numbers of targets to be detected in a multiplex reaction. In some embodiments, the sensitivity of the isothermal amplification is dramatically enhanced such that hundreds to thousands to hundreds of thousands of targets may be detected in a multiplex reaction. In some embodiments, the presence of the recombinant nucleic acid primer enhances the sensitivity of the isothermal amplification process by from 106 to 107 fold amplification of signal when combined with a multiplexed assay. In some embodiments, the false positive rate of a multiplexed assay is substantially reduced or zero.

### III. A Nucleic Acid Amplification Process

One aspect of the present disclosure provides a nucleic acid amplification process comprising a recombinant nucleic acid primer as described herein.

In some embodiments, the nucleic acid amplification process is an isothermal amplification selected from Loop-mediated isothermal amplification (LAMP or Loopamp); strand displacement amplification (SDA); recombinase polymerase amplification (RPA); helicase dependent amplification (HDA); Isothermal Multiple Displacement Amplification (IMDA); Rolling Circle Amplification (RCA); signal-mediated amplification of RNA technology (SMART); polymerase spiral reaction (PSR); or nicking enzyme amplification reaction (NEAR). In some embodiments, the nucleic acid amplification process is recombinase polymerase amplification (RPA); strand displacement amplification (SDA); or helicase dependent amplification (HDA). In one embodiment, the nucleic acid amplification process is recombinase polymerase amplification (RPA). In one embodiment, the nucleic acid amplification process is a homologous recombination enzyme directed isothermal amplification comprising a recombinase enzyme.

Isothermal amplification techniques were recently developed and are playing a significant role in the diagnosis of multiple diseases. Isothermal amplification techniques include, but are not limited to Loop-mediated isothermal amplification (LAMP or Loopamp); strand displacement amplification (SDA); recombinase polymerase amplification (RPA); helicase dependent amplification (HDA); Isothermal Multiple Displacement Amplification (IMDA); Rolling Circle Amplification; polymerase spiral reaction (PSR); signal-mediated amplification of RNA technology (SMART); and nicking enzyme amplification reaction (NEAR). See, Li & Macdonald, (Biosens. Bioelectron. 64:196-211 (2015)); Pumford et al., Biosens Bioelectron. 170:112674 (2020); Gill & Ghaemi, Nucleosides Nucleotides & Nucleic Acids 27 (3): 224-43 (2008).

Some isothermal amplification techniques rely on the biological activity of enzymes involved during homologous recombination (i.e., DNA repair, recombinases and accessory molecules), DNA replication (i.e., helicases and accessory molecules), or a restriction enzyme to melt the target double stranded DNA at a desired region (e.g., a nucleic acid region that is complementary to one or more oligonucleotide that is present in the amplification); specific polymerases (DNA and RNA polymerase; strand-displacing DNA polymerase); and/or specially designed primers (a single primer, primer pairs, multiple primer sets, or DNA-RNA chimeric primers).

In some embodiments, the nucleic acid amplification process comprising a recombinant nucleic acid primer as described herein is selected from rolling circle amplification (RCA), loop-mediated isothermal amplification, a Loop-mediated isothermal amplification (LAMP or Loopamp); strand displacement amplification (SDA); recombinase polymerase amplification (RPA); helicase dependent amplification (HDA); signal-mediated amplification of RNA technology (SMART); polymerase spiral reaction (PSR); nicking enzyme amplification reaction (NEAR). As is apparent to the skilled artisan, each isothermal amplification technique has its own preferred combination of enzymes and components that allow efficient and selective amplification of a target and/or targets, and/or internal control nucleic acids and are known to the skilled artisan.

Some isothermal amplification techniques rely on the biological activity of enzymes involved during homologous recombination (i.e., DNA repair, recombinases and accessory molecules), DNA replication (i.e., helicases and accessory molecules), or a restriction enzyme to melt the target double stranded DNA at a desired region (e.g., a nucleic acid region that is complementary to one or more oligonucleotide that is present in the amplification); specific polymerases (DNA and RNA polymerase; strand-displacing DNA polymerase); and/or specially designed primers (a single primer, primer pairs, multiple primer sets, or DNA-RNA chimeric primers).

These isothermal amplification techniques do not require thermal melting of DNA or any thermostable components. Rather, isothermal amplification techniques are performed at a single and constant temperature, using the biological activity of enzymes rather than temperature changes to copy nucleic acid sequences.

### 1. Loop-Mediated Isothermal Amplification (LAMP or Loopamp)

In some embodiments, the amplification reaction contemplated by the present disclosure is an isothermal amplification, such as Loop-mediated isothermal amplification (LAMP or Loopamp). LAMP provides a good alternative to PCR-based amplification assays. See e.g., Notomi et al., Nucleic Acids Res. 28: E6 (2000). LAMP uses a strand-displacing DNA polymerase and four or six primers to provide a very fast and specific amplification of target DNA or cDNA at a constant temperature based on the formation of an intermediate single strand nucleic acid product that comprising hairpin loops at each end (Loopamp Starting Structure). Loopamp Starting Structure has a dumbbell structure. Loopamp Starting Structure is a seed for the exponential LAMP amplification phase because it contains multiple sites for initiation of synthesis from the three prime ends of the open loops and annealing sites for both the inner and loop primers. The resulting products of a LAMP are long concatemers that are easily detected.

One key advantage of LAMP is its speed. LAMP is a very fast approach to synthesizing a lot of DNA in a very short period of time, because the Loopamp Starting Structure and the concatamers have multiple DNA synthesis initiation site. LAMP is currently used as a rapid molecular tests to detect microorganisms and or biomarkers associated with various biological disorders. However, during the COVID-19 pandemic, it was observed that, when used as part of a rapid COVID-19 diagnostic assays, LAMP generated up to 20% false negative results in patients with low SARS-COV-2 viral load. Butler et al., (bioRxiv doi: 10.1101/2020.04.20.048066). The sensitivity of LAMP as an assay tool for COVID-19 to detect SARS-COV-2 was efficient only when more than a few hundred copies of SARS-COV-2 viral RNA was present in the reaction assay. See e.g., Ben-Assa et al., medRxiv 2020.04.22.20072389); Zhang et al, medRxiv. doi: 10.1101/2020.02.26.20028373.

### 2. Helicase-Dependent Amplification (HDA)

In some embodiments, the amplification reaction contemplated by the present disclosure is an isothermal amplification, such as a helicase dependent amplification (HAD) reaction. HDA is an isothermal amplification process that uses the biological activity of a helicase to invade and unwind the double stranded DNA (dsDNA) molecule to initiate the amplification process. This isothermal technique is based on the biological activity of enzymes involved during in vivo DNA replication process. The helicase is involved in the repair of mismatches in DNA, and normally works in coordination with methyl-directed mismatch repair (MutL) protein that stimulates the helicase activity. As such, the original HDA system requires, the helicase (e.g., E. coli UvrD), and two accessory proteins: MutL and a single-stranded DNA-binding protein (SSB) to unwind the dsDNA and to stabilize the ssDNA preventing the re-association of the complementary strands. Once the dsDNA is isothermally unwound and stabilized, two oligonucleotide primers hybridize to the 5′ and 3′ borders of the target sequence and a DNA polymerase extends the annealed primers by adding deoxynucleotide-triphosphates (dNTPs) to generate double-stranded amplification products. The helicase-catalyzed reaction is the rate-limiting step. In addition, the helicase and polymerase must work in a coordinated way to prevent the polymerase to be displaced by the helicase.

DA has been used for the detection of viral and bacterial protein in biological samples. See, Barreda-García et al., (Analytical and Bioanalytical Chemistry 410:679-693 (2018)). However, the utility of HDA is hampered by a high rate of nonspecific amplification phenomenon that gives rise to false positive results. Some of the high rate of false positive is attributed to template-independent primer interactions and adversely affects the detectability and robustness of HDA. In some embodiments, the SDA further comprises a recombinase enzyme that binds to and coats the recombinant nucleic acid primer as disclosed herein. In some embodiments, the addition of the recombinase nucleic acid of the present disclosure enhances the sensitivity and efficiency of the HAD reaction and reduces nonspecific amplification products and false positive rate.

### 3. Strand Displacement Amplification (SDA)

In some embodiments, the amplification reaction contemplated by the present disclosure is an isothermal amplification, such as strand displacement amplification (SDA). Sec e.g., Walker et al., Proc. Natl. Acad. Sci. Unit. States Am. 89:392-396 (1992); Walker et al., Nucleic Acids Res. 20:1691-1696 (1992). Because it has an initial denaturation step, SDA is not a true isothermal amplification reaction. SDA relies on a restriction enzyme (HincII), an exonuclease-deficient DNA polymerase, and four primers. SDA begins with a denaturation step in the presence of four primers. Two primers consist of complementary sequences to the target DNA at their 3′ end and restriction enzyme sites of HincII at their 5′ end. The other two primers are normal primers forward and reverse primers. The pre-heating step denatures the double-stranded DNAs, while the rest of the reaction is performed at 37° C. SDA requires (i) a restriction endonuclease to nick the unmodified strand of the DNA target and (ii) an exonuclease-deficient strand displacing DNA polymerase (e.g., exo-Klenow polymerase) to displace the downstream DNA strand at the nick sites. The two enzymes are added after the initial denaturing step. As polymerase synthesizes DNA, newly synthesized strands are nicked by a restriction enzyme and polymerase starts synthesis again, kicking off the previous strand, resulting in DNA amplification. Some limitations includes lower primer specificity.

In some embodiments, the SDA comprises a recombinant nucleic acid primer of the present disclosure. In some embodiments, the SDA further comprises a recombinase enzyme that binds to and coats the recombinant nucleic acid primer as disclosed herein. In some embodiments, the addition of the recombinant nucleic acid primer and the recombinase obviates the need for an initial heat-dependent denaturation step. In some embodiments, the recombinant nucleic acid primer and the recombinase enhances primer specificities, thereby reducing non-specific amplification products and reducing false positive rate (FPR).

### 3. Isothermal Multiple Displacement Amplification (IMDA)

In some embodiments, the amplification reaction contemplated by the present disclosure is an isothermal amplification, such as Isothermal Multiple Displacement Amplification (IMDA). IMDA amplifies nucleic acid sequences using a strand-displacing DNA polymerase and multiple primer sets. Gill & Ghaemi, Nucleosides Nucleotides & Nucleic Acids 27 (3): 224-43 (2008). One advantage of IMDA over PCR is its sensitivity and specificity when the amount of DNA in the sample is very low. Mairinger et al., Dovepress 2014:7, 1441-1447.

### 4. Rolling Circle Amplification (RCA)

In some embodiments, the amplification reaction contemplated by the present disclosure is an isothermal amplification, such as Rolling Circle Amplification (RCA). RCA synthesizes long single-stranded DNA using a short, circular single-stranded DNA template and a single primer. RCA enzymatically synthesizes DNA using a strand displacing DNA polymerase (e.g., phi 29 (Φ29) DNA polymerase). RCA has been used for sensitive DNA detection in genomics and diagnostics. Zhao et al., Angew Chem. Int. Ed. Engl. 47 (34): 6330-7 (2008).

### 5. Recombinase Polymerase Amplification (RPA)

In some embodiments, the amplification reaction contemplated by the present disclosure is an isothermal amplification reaction that does not require a thermocycle, a thermostable DNA or RNA polymerase to amplify DNA, or heat to denature the double stranded DNA to facilitate the annealing of the reverse and forward primers to their complementary nucleic acid sequences. Rather, the isothermal amplification of the present disclosure is an improved and alternative amplification process that utilizes enzymes involved in the cellular DNA replication and repair machinery (e.g., homologous pairing or D-loop formation), such as the homologous recombination (U.S. Pat. Nos. 5,223,414; 6,929,915). In some embodiments, the isothermal amplification reaction is a recombinase polymerase amplification (RPA).

During homologous recombination in bacteria, such as E. coli, or various strain of bacteriophages, the bacterial recombinase protein (RecA), or its prokaryotic and eukaryotic relatives (such as UvsX), binds to and coat single-stranded DNA (ssDNA) to form a nucleoprotein filament. This nucleoprotein filament then scans the target double-stranded DNA (dsDNA) to identify a region that is homologous to the single-stranded DNA. When the homologous sequence is located, the nucleoprotein filament strand invades the dsDNA to create an hybrid DNA bubble call the D-loop. The D-loop comprises: (1) a short double-strand hybrid between the ssDNA and one of the displaced strand of the dsDNA (hybrid filament), and (2) a displaced strand of DNA. In the presence of a DNA polymerase, the free 3′-end of the hybrid filament strand in the D-loop is extended by DNA polymerases to synthesize a new complementary strand. The complementary strand displaces the originally paired strand as it elongates. This entire process occurs without the need to completely denature the entire double stranded DNA molecule.

Akin to the recombination system, the RPA process utilizes a recombination factor derived from a bacteria or bacteriophage (e.g., a recombinase UvsX) to catalyze the invasion and limited denaturation of the target double-stranded DNA to facilitate the hybridization of the forward and reverse primers (e.g. ssDNA). In particular, the first step usually requires the binding of the recombinase (recombinase factor) derived from E. coli or a bacteriophage with the forward and reverse primers to form a recombinase-primer complex (nucleoprotein complex). This is followed by a recombinase-mediated primer insertion into the double stranded DNA with help of at least two accessory proteins: a recombinase loading factor (e.g., bacteriophage UvsY) and/or a single stranded DNA binding protein (e.g., SSB, or gp32) that facilitates the isothermal amplification process. The forward and reverse primers then individually hybridizes to their respective homologous sequences. When the DNA polymerase is recruited to the sites, it initiates the amplification of the target nucleic acid sequence.

However, as known to those of skill in the art, previously established isothermal amplification reactions have not been optimal for clinical use for a number of reasons. See e.g., U.S. Pat. Nos. 8,071,308 and 7,399,590. One of the key reasons is the well established competition between the recombinase enzyme (e.g., UvsX) and the single-stranded binding protein (SSB; gp32.) for the binding of the single stranded nucleic acid such as the reverse and forward primer. While the SSB protein is essential in the in vitro isothermal amplication process, the SSB generally has a considerably higher affinity for a single-stranded DNA (e.g., primers) than the recombinase (e.g., RecA or UvsX). As such, SSB can inhibit the nucleation of recombinase/primer nucleoprotein complex. Moreover, the nucleoprotein complex is unstable in the presence of ATP and undergoes end-dependent disassembly (Bork, Cox and Inman, J Biol Chem. 2001 Dec. 7; 276 (49): 45740-3). After such a disassembly event, the SSB rapidly saturates unbound single strand nucleic acid and inhibits the initiation of the invasion reaction.

This competition was dealt with in a number of ways including (1) the addition of a recombinase loading factor (e.g., UvsY) that is specific for the recombinase (e.g., UvsX); engineering recombinase proteins (e.g., UvsX) and/or SSB mutants with enhanced cooperative behavior; the addition of crowding agents to the reaction; and or the introduction of a non-functional third primer to obviate the competition between the recombinase and the SSB. However, these changes did not generate an improved isothermal amplification process with high sensitivity, efficiency, and reduced false positive rate (FPR).

### 6. Invasion Oligonucleotide and Self-Templatization

One aspect of the present disclosure provides a highly sensitive and efficient isothermal amplification process for disease diagnosis and prognosis (e.g., odetection of SARS-CoV-2 for the diagnosis of COVID-19 infection). The novel and improved isothermal amplification reaction uses a recombinant nucleic acid primer in conjunction with a wild-type enzyme (i.e., recombinase, and/or helicase), a single-stranded binding protein and does not require a recombinase loading factor. The novel recombinant nucleic acid primer is a substantial improvement over the invasion oligonucleotide primer known in the art. In some embodiments, the recombinant nucleic acid primer is a long, non-extendable oligonucleotide that acts as a nonfunctional primer to facilitate the invasion and unwinding of the dsDNA template.

The invasion oligonucleotide primer (IO) is about 39-61 bases in length and contained three 3 sub-regions. These three sub-regions, listed from 5′ to 3′ were: (1) a poly-dC sub-region with about 7-13 bases in length; (2) a DNA sub-region that was homologous to the target nucleic acid region and was about 25-35 bases in length; (3) a methylated RNA sub-region that is also homologous to the target nucleic acid region, and (4) a 3′-end inverted nucleotide base modification. The methylated RNA sub-region was designed to ensure that the primers did not anneal to it and the polymerase did not use it as a substrate for amplification. The poly-dC sub-region was designed to be a seeding region that optimized the binding of the recombinase the nucleic acid recombinase. The DNA subregion was designed to facilitate the invasion and unwinding of the target dsDNA to permit the partial denaturing of the target dsDNA.

During an isothermal amplification, the recombinase (UvsX) and the IO primer; binds to each other to form a nucleoprotein complex. This nucleoprotein complex then searches the target nucleic acid for homologous sequence. Once found, the nucleoprotein complex invades the complementary region of a dsDNA target unwinds the dsDNA into single strands and facilitates the hybridization of the forward and reverse primers to the template DNA. The primers then bind to their respective complementary sites on each end of a template double stranded DNA (dsDNA). Following the hybridization of the primers to the template, the polymerase and SSB (e.g., T4gp32) are recruited to the sites. SSB (e.g., T4gp32) stabilizes the unbound DNA strand and transiently maintains the two single strands of DNA apart, thereby facilitating DNA synthesis by the polymerase. The polymerase replicates and amplifies the target sequence by adding dNTPs to each of the two primers to generate an amplified copy. In this system, the primers were too short (normal PCR primer size, 18-25 bases) to be bound by a recombinase.

Despite the presence of the methylated RNA sub-region (2′-O-methyl RNA), experimental evidence by the Applicant showed that the recombinant nucleic acid primer could itself function as template for the forward and/or reverse primers and polymerase during the amplification process. Indeed a high rate of false positive results were obtained when the IO primer of the art was used. Indeed, both the forward and reverse primer annealed to the IO primer, which was amplified by a polymerase. A schematic illustration of a non-specific amplicon generated by the IO primer known in the art is shown in FIG. 2.

Isothermal amplication using the IO primer resulted in a high rate of false positive in diagnostic tests (e.g., COVID-19 PCR test). The rate of non-specific amplification was exacerbated when the recombinant nucleic acid primer was used in the multiplexed assay system where the multiplicity of the assay was increased, and/or additional primers was required. Under these circumstances, the probability of non-specific primer-to-primer interactions was generally increased. Sequencing of the non-specific amplification products indicated that the polymerase was still able to use the recombinant nucleic acid primer as a substrate and/or template despite the presence of the methylated sub-region (2′-O-methyl RNA), which resulted in self-templating amplification. The self-templating amplification was a particular issue when introducing the recombinant nucleic acid primer because the recombinant nucleic acid primer contains much of the target region of interest. Therefore, copies of the recombinant nucleic acid primer were made via-self templatization rather than the desired target template. Furthermore, the recombinant nucleic acid primer was designed to have an overlapping region with the forward primer and reverse primer. This design feature also enhanced self-templatization. As used herein, the term “self-templatization” means that during the isothermal amplification process, the amplification product was generated by using the recombinant nucleic acid primer as a template for amplification rather than the target template.

The Applicant accordingly hypothesized that the high rate of false positive results when using the original recombinant nucleic acid primer could be prevented by redesigning the recombinant nucleic acid primer to enhance the sensitivity, and efficiency of the isothermal amplification, and to eliminate and/or reduce False positive rate. The present disclosure provides an optimal design for a recombinant nucleic acid primer for use in a multiplex assay that exhibits low background characteristics, while maintaining exceptional functionality for template-dependent amplification. In some embodiments, the recombinant nucleic acid primer (modified invasion oligonucleotide primer; modified IO primer; oligonucleotide) was designed not to bind to or bind weakly to a single stranded binding protein (SSB).

One aspect of the present disclosure provides a novel isothermal amplification that works effectively in the clinical application of the isothermal reaction (e.g., COVID-19 testing). The novel reaction uses a wild-type recombinase, a single-stranded binding protein and the recombinant nucleic acid primer described herein. In some embodiments, the novel isothermal amplification process uses a single step reaction that requires the simultaneous addition of: two specific primers (e.g., forward and reverse primers); recombinant nucleic acid primer (a long/non-extendable oligonucleotide, a nonfunctional primer; modified invasion oligonucleotide primer; modified IO primer; oligonucleotide) that was designed not to bind to or bind weakly to a single stranded binding protein (SSB); a recombination factor (e.g. UvsX) protein from a bacteriophage; a single stranded binding protein (SSB) from bacteriophage; a DNA polymerase; and a crowding agent, such as glycerol, ficoll, or a low molecular weight PEG to enhance the kinetics of the amplification reaction.

In some embodiments, the nucleic acid amplification process comprises the steps of: contacting a recombinase with the recombinant nucleic acid primer (e.g., IO primer; invasion oligonucleotide; or recombinant oligonucleotide) to form an oligo-recombinase complex; contacting the oligo-recombinase complex to the complementary double stranded target nucleic acid under conditions that allow the oligo-recombinase complex to invade the double stranded target nucleic acid; admixing a forward, a reverse primer, a polymerase, and deoxynucleotide triphosphates (dNTPs) to the amplification process under conditions that allow the forward and reverse primers to bind to their respective complementary regions on the double stranded target nucleic acid, and that allow the polymerase to extend the 3′ end of the forward and reverse primers with dNTPs to generate a first and second amplified double-stranded nucleic acid; and optionally continuing the amplification process through repetition of preceeding steps until a desired degree of amplification product is produced.

In some embodiments, the forward and revers primer used in the nucleic acid amplification process described herein is selected from a DNA, RNA, PNA, LNA, morpholino backbone nucleic acid, phosphorothioate backbone nucleic acid or a combination thereof.

In some embodiments, the oligo-recombinase complex invasion of the double stranded target nucleic acid denatures the region of the double stranded target nucleic acid that is complementary to the recombinant nucleic acid primer. In some embodiments of the nucleic acid amplification process described herein, the nucleic acid is amplified in the absence of a recombinase loading factor. In some embodiments of the nucleic acid amplification process described herein, the nucleic acid is amplified in the presence of a recombinase loading factor. In some embodiments, the recombinase loading factor is selected from a bacteriophage uvsY, an E. coli recO, an E. coli recR, derivatives thereof or a combination of these proteins.

In some embodiments of the nucleic acid amplification process described herein, the nucleic acid is amplified in the presence of a single stranded DNA binding molecule (SSB); and optionally the SSB is selected from E. coli SSB protein, a gp32 protein from any bacteriophage; a gp32 protein derived from a myoviridae bacteriophage, a T4 gp32 protein, Rb69 gp32 protein, or derivatives thereof. In some embodiments, the SSB stabilizes nucleic acids during the various exchange transactions that are ongoing in the amplification reaction. As described herein, the art understands that the nucleoprotein complex between the recombinase and the single-stranded DNA is unstable in the presence of a SSB protein. The present disclosure addresses this competition by including an IO primer that is distinct from the forward and reverse primers that are necessary for the amplification step of the reaction. In some embodiments, the SSB (gp32) SSB does not bind or binds weakly to the recombinant nucleic acid primer, thereby stabilizing or enhancing the formation of a recombinase-IO primer complex. In some embodiments, the presence of the IO primer negates the need of a recombinase loading factor during amplification.

In some embodiments of the nucleic acid amplification process described herein, the recombinase is selected from E. coli RecA, or a UvsX from any bacteriophage species. In some embodiments, the recombinase is derived from a myoviridae phage selected from T4, T2, T6, Rb69, Ach1, KVP40, Acinetobacter phage 133, Acromonas phage 65, cyanophage P-SSM2, cyanophage PSSM4, cyanophage S-PM2, Rb14, Rb32, Aeromonas phage 25, Vibrio phage nt-1, phi-1, Rb16, Rb43, Phage 31, phage 44RR2.8t, Rb49, phage Rb3, and phage LZ2. In some embodiments, the recombinase is T4 UvsX, Rb69 UvsX, T2 UvsX, T6 UvsX, Aeh1 UvsX, KvP40 UvsX, or any derivatives or any combinations thereof.

In some embodiments of the nucleic acid amplification process described herein, the polymerase is selected from E. coli Pol I, Bacillus subtilis Pol I large fragment (Bsu polymerase), Moloney Murine Leukemia Virus (MuMLV) reverse transcriptase, Staphylococcus aureus Pol I, or a combination thereof.

In some embodiments of the nucleic acid amplification process described herein, the amplification process further comprises the addition of a crowding agent selected from polyethylene glycols, PEG1450, PEG6000, PEG8000, PEG10000, PEG15000, PEG20000, PEG35000, glycerol, dextran, ficoll, or a combination thereof.

In some embodiments of the nucleic acid amplification process described herein, the recombinant nucleic acid primer, the target nucleic acid molecule, the forward and reverse primers, the polymerase, and dNTPs are added simultaneously to the nucleic acid amplification process.

In some embodiments, the recombinase and the SSB are from the same species; or the recombinase and the SSB are from different species. In some embodiments, the recombinase and the SSB are from the same bacteriophage species; or the recombinase and the SSB are from different bacteriophage species.

In some embodiments, the recombinase and the SSB are derived from a myoviridae bacteriophage, a T4 bacteriophage, Rb69 bacteriophage, T2 bacteriophage, T6 bacteriophage, Aeh1 bacteriophage, KvP40 bacteriophage, or derivatives thereof. In some embodiments, the recombinase and the SSB are: (a) Rb69 UvsX and Rb69 gp32, T4 gp32, or Aeh1 gp32, or T6 gp32; (b) Aeh1 UvsX, and Rb69 gp32, T4 gp32, or Aeh1 gp32, or T6 gp32; (c) T4 UvsX, and Rb69 gp32, T4 gp32, or Aeh1 gp32, or T6 gp32; and (d) T6 UvsX and Rb69 gp32, T4 gp32, or Aeh1 gp32, or T6 gp32. In some embodiments, the recombinase is Rb69 UvsX, and the SSB is T4 gp32. In some embodiments, the polymerase is Bacillus subtilis Pol I large fragment (Bsu polymerase), Moloney Murine Leukemia Virus (MuMLV) reverse transcriptase, or a derivative or combination thereof.

### IV. A Composition Comprising a Recombinant Nucleic Acid Primer

One aspect of the present disclosure provides a composition comprising one or more recombinant nucleic acid primer (i.e., oligonucleotide, Invasion Oligonucleotide, or “IO primer”) as described herein. In some embodiments, the composition comprises: (i) the recombinant nucleic acid primer (i.e., oligonucleotide, Invasion Oligonucleotide, or “IO primer”); (ii) a recombinase; (iii) a polymerase; (iv) a target nucleic acid; (v) a crowding agent; (vi) one or more primer; and (vi) a single stranded DNA binding protein (SSB).

In some embodiments, the composition is used in single or a multiplexed assay. When used in a multiplexed assay, a plurality of primers are combined each with a specificity to a distinct target nucleic acid region. In some embodiments, the presence of the recombinant nucleic acid primer in the composition significantly enhances the specificity and sensitivity of the multiplexed assay when compare to a multiplexed assay conducted in the absence of the recombinant nucleic acid primer. In some embodiments, the presence of the first and second methylated RNA regions on the recombinant nucleic acid primer as described herein enhances the specificity and sensitivity of a multiplexed assay when compared to a recombinant nucleic acid primer containing only the first or the second methylated RNA region.

### 1. A Single Stranded DNA Binding Protein (SSB)

In some embodiments, the composition as described herein comprises a SSB selected from E. coli SSB protein, a gp32 protein from any bacteriophage; a gp32 protein derived from a myoviridae bacteriophage, a T4 gp32 protein, Rb69 gp32 protein, or derivatives thereof. SSB are required during the in vitro isothermal amplification of DNA. SSB proteins are essential because: (1) they bind to the single-stranded DNA that is not occupied by the forward and reverse primer; (2) they melt secondary structures within the double stranded DNA; (3) they facilitate the outgoing strand displacement by the DNA polymerase by binding to the newly release single strand of DNA; (4) they suppress branch migration during DNA amplification. The SSB is needed for binding and stabilizing the outgoing strand, and for melting secondary structures in single-stranded DNAs to enhance the processivity and synthetic activity of a polymerase. As noted above, SSB does not discriminate between a single-stranded DNA in the context of a replication fork (e.g., D-loop formation) and a forward and/or reverse primer in vitro.

### 2. Recombinase

In some embodiments, the composition as described herein comprises a recombinase selected from E. coli RecA, a UvsX from any bacteriophage species, a UvsX from a myoviridae bacteriophage, T4 UvsX, Rb69 UvsX, T2 UvsX, T6 UvsX, Ach1 UvsX, KvP40 UvsX, or any derivatives or combinations thereof.

A recombinase is a protein that promotes the formation of heteroduplex DNA during homologous recombination. A recombinase function requires by binding and coating single-stranded DNA (ssDNA) to form a nucleoptorein complex or filament. This nucleoprotein complex or filament then scan double-stranded DNA (dsDNA) molecule to identify the regions of sequence homology with the bound ssDNA. The location of a homologous duplex by the recombinase nucleoprotein/filament results in the invasion (opening) of the double stranded DNA, and the formation of a homologous joint between the ssDNA and the double stranded DNA. This in turn leads to strand exchange, during which, the ssDNA contained within the nucleoprotein filament forms Watson-Crick base pairs with the complementary strand of the “target” duplex, displacing the noncomplementary strand in the duplex. The hybrid DNA formed by strand exchange is further processed by repair polymerase and other recombination factors, eventually yielding two intact DNA duplexes. See e.g., Gasior et al., PNAS 98 (15): 8411-8418 (2001).

The most well characterized recombinases are the recombinases of the RecA family, including RecA in eubacteria, RadA in archea, Rad51 and Dmc1 in eukaryea, and the bacteriophage T4 UvsX protein. In some embodiments, a recombinase of the RecA family is derived from a bacteriophage, such as a myoviridae bacteriophage. In some embodiments, the recombinase is derived from one of the six genera of the Myoviridae family. In some embodiments, the bacteriophage recombinase is a UvsX molecule derived from T4 bacteriophage, T2 bacteriophage, T6 bacteriophage, Rb69 bacteriophage, Ach1 bacteriophage, KVP40 bacteriophage, Acinetobacter phage 133, Acromonas phage 65, cyanophage P-SSM2, cyanophage PSSM4, cyanophage S-PM2, Rb14, Rb32 bacteriophage, Acromonas phage 25, Vibrio phage nt-1, phi-1, Rb16, Rb43, Phage 31, phage 44RR2.8t, Rb49, phage Rb3, and phage LZ2.

In some embodiments, the recombinase is derived from a bacteriophage that belongs to the genus of T4-like bacteriophages, which includes at least T4, T2, T6, Rb69, Ach1, KVP40. The T4-type of bacteriophages are broadly defined on the basis of particle morphology. They occur in enterobacteria (125 representatives), acinetobacters, acromonads, pseudomonads, and vibrios (16 isolates).

In some embodiments, the recombinase is Rb69 UvsX. The art discloses that Rb69 UvsX does not function in the same way as other UvsX proteins of the T4-like bacteriophages family. See eg., U.S. Pat. No. 8,071,308. RB69 UvsX has a relatively poor DNA binding, and does not support the hybridization of complementary oligonucleotides. The art discloses that Rb69 UvsX has a low affinity for, or residence time on ssDNA, compared with T4 UvsX or T6 UvsX which means that it competes poorly with excess gp32 (hence sensitivity to gp32 overtitration). Rb69 UvsX also fails to support oligonucleotide hybridizations and thus encouraging overly high oligonucleotide-recombinase loading also leads to impaired amplification reactions as few primers are available for hybridization. Structurally, Rb69 UvsX is also different because it has how very unusual loop2 sequence when compared to its nearest homologous neighbors, such as T4, T6, Ach1, KVP40, phage 133. The loop 2 of Rb69 UvsX also has a different number of amino acids and appears completely recoded in comparison to T4, T6, Ach1, KVP40, phage 133 (and all UvsX molecules apart from JS98 which is a close Rb69 relative), and the cyanophage proteins.

In fact, significant efforts were made to get an amplification reaction to work using the RB69 UvsX in vitro. In particular, an isothermal amplification reaction are sensitive to the concentrations of the recombinase RB69 UvsX and/or RB69 gp32. Moreover, the recombinase UvsX derived from RB69 could not function in vitro in the absence of a recombinase loading protein, such as UvsY. See eg., U.S. Pat. No. 8,071,308.

Despite all the difficulties disclosed in the art regarding the use of the recombinase UvsX derived from the bacteriophage Rb69, the Applicant surprisingly found that Rb69 UvsX worked extremely well in the claimed composition and methods. Rb69 UvsX did not require a recombinase loading factor. The efficacy of the Rb69 UvsX in the methods and compositions described herein are attributed to the presence of the recombinant nucleic acid primer (i.e., oligonucleotide, Invasion Oligonucleotide, or “IO primer”) as described herein. Indeed, the recombinant nucleic acid primer described herein simplified the isothermal reaction and enhance the amplification kinetics by invading and denaturing a larger region of the double stranded DNA. Moreover, the compositions and methods described herein are more similar to PCR where the sole function of the forward and reverse primers is to prime the exposed single-stranded DNA for amplification by a DNA polymerase.

In some embodiments, the recombinase and the SSB are from the same species; or the recombinase and the SSB are from different species. In some embodiments, the recombinase and the SSB are from the same bacteriophage species or the recombinase and the SSB are from different bacteriophage species. In some embodiments, the recombinase and the SSB are derived from a myoviridae bacteriophage, a T4 bacteriophage, Rb69 bacteriophage, T2 bacteriophage, T6 bacteriophage, Ach 1 bacteriophage, KvP40 bacteriophage, or derivatives thereof.

In some embodiments of the composition as described herein (a) the recombinase is Rb69 UvsX and the SSB is selected from Rb69 gp32, T4 gp32, Ach1 gp32, or T6 gp32; (b) the recombinase is Ach1 UvsX, and the SSB is selected from Rb69 gp32, T4 gp32, Ach1 gp32, T6 gp32, or derivative thereof; (c) the recombinase is T4 UvsX, and the SSB is selected from Rb69 gp32, T4 gp32, Ach1 gp32, T6 gp32, or any derivative thereof; or (d) the recombinase is T6 UvsX and the SSB is selected from Rb69 gp32, T4 gp32, Ach1 gp32, T6 gp32, or any derivative thereof. In some embodiments of the composition as described herein, the recombinase is Rb69 UvsX, and the SSB is T4 gp32.

### 3. Recombinase Loading Factor

In some embodiments of the composition as described herein, the composition is free of a recombinase loading factor. In some embodiments of the composition as described herein, the composition comprises a recombinase loading factor. In some embodiments, a recombinase loading factor is selected from bacteriophage uvsY, E. coli recO, or E. coli recR and derivatives and combinations thereof. As described herein, the art discloses that a recombinase loading agent (e.g., UvsY) (1) facilitated the binding of recombinase (UvsX) with the single nucleic acid primer to form a nucleoprotein complex, (2) dealt with the competition between gp32 and the recombinase for binding to single stranded DNA (i.e. primers); (3) stabilized the nucleoprotein complex for D loop creation; and/or (4) increased recombinase loading in an unfavorable SSB/gp32 environment. In some embodiments of the composition as described herein, the presence of the IO primer negates the need for a recombinase loading factor during the amplification process.

### 4. Polymerase

In some embodiments of the composition as described herein, the polymerase is selected from E. coli Pol I, Bacillus subtilis Pol I large fragment (Bsu polymerase), Moloney Murine Leukemia Virus (MuMLV) reverse transcriptase, Staphylococcus aureus Pol I, or a combination thereof. In some embodiments, the polymerase is Bsu polymerase, MuMLV, and/or a derivative thereof.

Additional polymerases that may be used with the composition described herein include E. coli DNA polymerase I Klenow fragment, bacteriophage T4 gp43 DNA polymerase, Bacillus stearothermophilus polymerase I large fragment, Phi-29 DNA polymerase, T7 DNA polymerase, Bacillus subtilis Pol I, E. coli DNA polymerase I, E. coli DNA polymerase II, E. coli DNA polymerase III, E. coli DNA polymerase IV, E. coli DNA polymerase V and derivatives and combinations thereof.

In some embodiments, the polymerase is a strand displacing polymerase. In strand-displacement replication, only one strand is replicated at once. This synthesis releases a single stranded DNA, which is in turn copied into double strand-DNA. During strand displacement, a polymerase opens the double-stranded (ds) DNA in order to extend a primer. The synthetic and strand-displacing activities of a strand-displacing DNA polymerase results in the production of a double-stranded DNA and a displaced single strand. This displaced single strand is replicated either by direct hybridisation and elongation of a second oligonucleotide/primer, or by strand displacement synthesis if an invasion event had already occurred from the opposite end.

While many polymerases exhibit rapid and processive primer extension activity, inefficient strand displacement activity seems to be a general feature of replicative polymerases. As a consequence, replicative helicases are required to open the duplex DNA and facilitate the advancement of the leading-strand polymerase in the context of a replisome. In the present disclosure, replicative helicases are not required to open the duplex DNA and facilitate the advancement of the leading-strand polymerase because of the presence of the recombinant nucleic acid primer (i.e., oligonucleotide, Invasion Oligonucleotide, or “IO primer”). In some embodiments, the single-strand binding protein (e.g., E. coli SSB or bacteriophage gp32) is necessary to enable efficient strand displacement during DNA amplification. In some embodiments, the polymerase is not a strand displacing polymerase.

In some embodiments, the polymerase has high processivity. As used herein, the term “processivity” means the ability of DNA polymerase to carry out continuous DNA synthesis on a template DNA without frequent dissociation. Processivity is measured by the average number of nucleotides incorporated by a DNA polymerase on a single association/disassociation event. DNA polymerase's processivity reflects the synthesis rate and speed, as well as affinity for its substrates. Therefore, highly processive DNA polymerases are beneficial for amplification of long templates and of sequences with secondary structures and high GC content, and in the presence of PCR inhibitors such as heparin, xylan, and humic acid, which are found in blood and plant tissues. Zhuang et al., Biochim. Biophys. Acta 1804 (5): 1081-1093 (2010). In some embodiments, the polymerase has high processivity is selected from Phi-29 DNA polymerase, Bst DNA polymerase, Bacillus subtilis Poll (Bsu), and T7 DNA polymerase.

### 5. Crowding Agent

In some embodiments of the composition as described herein, the composition comprises a crowding agent. A crowding agent aids in establishing an optimal amplification reaction environment. Macromolecular crowding agents such as Ficoll, Dextran, and polyethylene glycol, increase the reaction speed of several enzymes in vitro. In particular, crowding agents increases the activity of DNA polymerases, RNA polymerases, ligases, endonucleases, and exonucleases. Crowding agents increase the rate of nucleotides incorporation during DNA amplification by stabilizing DNA-polymerase complex. See e.g., Zimmerman and Harrison, Proc. Natl. Acad. Sci. USA, 84:1871-1875 (1987); Ellis et al., Curr. Opin. Struc. Biol. 11 (1): 114-119 (2001); Ikeda et al., U.S. Pat. No. 5,665,572. For example, macromolecular crowding causes significant increase in the binding of DNA polymerase I (Pol 1) of Escherichia coli or the T4 DNA polymerase to their template primers and/or DNA. In particular, the addition of a crowding agent, such as polyethylene glycol (PEG8000 and PRG35000) enhanced the binding affinity and activity of the DNA Pol I and T4 DNA Pol I in vitro. Crowding agents also enhance the exonuclease and polymerase activities of E. coli DNA Pol I and the T4 DNA polymerase.

In some embodiments of the composition as described herein, the crowding agent is selected from polyethylene glycols, PEG1450, PEG6000, PEG8000, PEG10000, PEG15000, PEG20000, PEG35000, glycerol, dextran, ficoll, or a combination thereof. In a preferred embodiments, the composition comprises PEG6000. In some embodiments, the composition comprises at least about 1% to at least about 12% by volume of a crowding agent per reaction. In some embodiments, the composition comprises at least about 1% to at least about 12% by weight of a crowding agent per reaction.

In some embodiments of the composition as described herein, the composition further comprises dNTPs. dNTPs are well known to those skill in the art. The dNTPs includes, for example, dATP, dGTP, dCTP, dTTP, and derivatives and analogs thereof. In some embodiments of the composition as described herein, the composition comprises NTPs selected from ATP, GTP, CTP, UTP, or derivatives and analogs thereof. In some embodiments, the composition comprises ddNTPs (ddATP, ddTTP, ddGTP and ddGTP) to generate e.g., fragment ladders. In some embodiments, the composition comprises a mixture of dNTPs and ddNTPs.

### 6. Dried Composition-Trehalose

An advantage of the isothermal amplification method described herein is that it does not require a sophisticated PCR machine, and the composition comprising the necessary reagents does not need refrigeration. This is possible because the composition may be dried and reconstituted with a liquid when ready to use. Freeze dried compositions can maintain their activity in the absence of refrigeration and can be maintained at room temperature for an unlimited time period. Excipient stabilizers may be added to the dried composition to enhance the shelf life of the dried composition and/or to improve the freeze-drying performance. The reagents that can be freeze-dried before use are the recombinase, the single stranded DNA binding protein, the DNA polymerase, the dNTPs or the mixture thereof, buffer, and the first primer.

In some embodiments of the composition as described herein, the composition is dried. In some embodiments, the crowding agent is not included in the dried composition. In some embodiments, the composition is freeze-dried, lyophilized powder, a pellet, or a bead. In some embodiments, the composition is lyophilized in a reagent ball or in a tube. In some embodiments, the composition is dried in the presence of an excipient stabilizer (e.g.; a protein stabilizer). In some embodiments, the excipient stabilizer is selected from buffers (e.g., phosphate, acetate, and histidine); tonicity agents/stabilizers (e.g., sugars such as sucrose, polyols such as sorbitol); bulking agents e.g., lyoprotectants such as mannitol); surfactants (e.g., polysorbates); antioxidants (e.g., methionine); metal ions/chelating agents (e.g., ethylenediaminetetraacetic acid, EDTA); or preservatives (e.g., benzyl alcohol). In some embodiments, the excipient stabilizer is a sugar selected from disaccharide sugars (e.g., sucrose, trehalose, maltose, and lactose) or polyols (e.g., mannitol, sorbitol, and glycerol). In some embodiments, the excipient stabilizer is trehalose. Trehalose is a naturally occurring osmolyte that is known to be an exceptional stabilizer of proteins and helps retain the activity of enzymes in solution as well as in the freeze-dried state. Trehalose is a universal stabilizer of protein conformation because of its exceptional effect on the structure and properties of solvent water when compared with other sugars and polyols. See e.g., Kaushik and Bhat, J. Biol. Chem. 278 (29): P26458-26465 (2003).

In some embodiments, the composition is reconstituted with water, a liquid, a buffer solution. In some embodiments, the reconstitution liquid comprises a crowding reagent, a target nucleic acid, a sample comprising a target nucleic acid. In some embodiments, the reconstituted composition is incubated until a desired degree of amplification product is produced.

### 7. Primers

In some embodiments of the composition as described herein, the composition comprises one or more primer set. In some embodiments, each primer set comprises a forward primer and a reverse primer. In some embodiments, each primer set amplifies a different target nucleic acid. In some embodiments, each primer set amplifies a different region of the same target nucleic acid

In some embodiments, the primer length are shorter than the length of primers used in the art. While primer length as short as 15 nucleotides have been shown to assemble functional homology-searching complexes with E. coli recA in the presence of the non-hydrolysable cofactor analogue ATP-γ-S, but a nucleoprotein complex formed by such a short primer could not function. In particular, the homology-searching function of the recombinase was not sufficient to complete strand exchange and to be released from the invasion complex to allow polymerase access. The art recommends a primer length at least about 30 bases for isothermal amplification. Salinas et al., J Biol Chem. 270 (10): 5181-6 (1995); Formosa et al. J. Biol. Chem. 261 (13): 6107-18 (1986). Furthermore, homology lengths as short as 33 nucleotides was sufficient to direct recombinase/ssDNA nucleoprotein complex to its appropriate targets i and to permit complete strand exchange. See eg., U.S. Pat. No. 7,399,590. The recommended optimal length of oligonucleotide for isothermal amplication lays between 30 nucleotides and 50 nucleotides, and that progressively larger oligonucleotides can decrease the rate of invasion/extension.

In contrast, the primer of the claims disclosures have the length of a routine PCR primer. Because the at least one primers of the present composition are not being used to assist the recombinase with the the homology-searching function, invasion, or strand exchange, they do not need to be longer than the optimal length of a PCR primer. In some embodiments, the primers are at least about 15 to at least about 25 bases. In some embodiments, the primers are at least about 16, at least about 17, at least about 18, at least about 18, at least about 19, at least about 20, at least about 21, at least about 22, at least about 23, at least about 24, at least about 25 bases. In some embodiments, the reverse or forward primer comprises DNA, RNA, peptide nucleic acids (PNA), locked nucleic acid (LNA), morpholino backbone nucleic acid, phosphorothiorate backbone nucleic acid or a combination thereof. As used herein, Peptide nucleic acid (PNA) means synthetic mimics of DNA in which the deoxyribose phosphate backbone is replaced by a pseudo-peptide polymer to which the nucleobases are linked. PNAs hybridize with complementary DNAs or RNAs with remarkably high affinity and specificity, essentially because of their uncharged and flexible polyamide backbone. As used herein, Locked nucleic acid (LNA) means a nucleic acid analogue containing one or more LNA nucleotide monomers with a bicyclic furanose unit locked in an RNA mimicking sugar conformation.

Various softwares for designing and generating primers are known to those skilled in the art and are commercially available. The primers of the disclosure are designed in accordance with methods that are known in the art. An initial consideration in choosing the length of primers is the temperature under which they will be expected to be utilized for the methods described herein. For example, the chosen length of an oligonucleotide might vary depending on the thermal stability of the cleavage means. Longer primers are generally expected to have a higher hybridization specificity. See Lyamichev et al. (1999) Nature Biotechnology 17:292-296; see also, U.S. Pat. Nos. 6,562,611; 5,994,069 and 5,846,717, the entire contents of which are hereby incorporated by reference. Furthermore, the primers of the present may be purchased from manufacturer such, Thermo Fisher Scientific, Integrated DNA Technologies, or Sigma-Aldrich.

### V. An Amplification Device

One aspect of the present disclosure provides an amplification device comprising the composition as described herein. In some embodiments, the device is used in a method for determining the presence, absence, or quantity of one or more target nucleic acids in a sample. In some embodiments, the device is used to amplify a nucleic acid using the recombinant nucleic acid primer, the composition and the isothermal amplification techniques described herein.

One aspect of the present disclosure provides an amplification device comprising a composition or system as described herein. In some embodiments, the device is used in a method for determining the presence, absence, or quantity of one or more target nucleic acids in a sample. In some embodiments, the method comprises or consists essentially of, or consist of: (i) amplifying (if present) the one or more target nucleic acids in the sample using the nucleic acid primer and/or system described herein; (ii) directly or indirectly binding the amplified target nucleic acids to one or more signaling agents and detecting the signaling agents if the target nucleic acids are present; (iii) detecting the amplified target amplified nucleic acids if present in the sample. If the target nucleic acids are not present in the sample, no signal is detected.

In some embodiments of the amplification device as described herein, the method for determining the presence, absence, or quantity of one or more target nucleic acids in a sample comprises: (i) introducing the sample comprising one or more target nucleic acids into fluid in a sample preparation reservoir of a sample analysis cartridge, wherein the cartridge comprises the composition as described herein; (ii) amplifying one or more target nucleic acids using the amplification process comprising the recombinant nucleic acid primer as described herein; (iii) directly or indirectly binding the amplified target nucleic acids to one or more signaling agents in the fluid in the sample preparation reservoir; (iv) releasing the fluid from the sample preparation reservoir into an analysis channel comprising a sensor such that the one or more amplified target nucleic acids directly or indirectly bind to the one or more signaling agents; (v) reacting a substrate with the signaling agent localized over the sensor; (vi) electrically stimulating the reacted substrate-signaling agent complex to generate one or more signals that are detected by the sensor; and (viii) processing the one or more signal to determine the presence, absence, or quantity of the one or amplified target nucleic acid.

In some embodiments, each one or more signaling agents in the fluid in the sample preparation reservoir has a detectable potential specific for each of the one or more amplified target nucleic acids. In some embodiments, the detectable potential for each one or more amplified nucleic acids is different. In some embodiments, the signal for each substrate-signaling agent complex is based on the respective potential of the signaling agent for the one or more amplified nucleic acid targets.

In some embodiments, the sensor simultaneously detects the one or more signals, thereby simultaneously detecting the presence, absence, or quantity of one or more target nucleic acids in a sample. In some embodiments, the device comprises more than one sensors.

In some embodiments, the device comprises a sample preparation reservoir. In some embodiments, the sample preparation reservoir comprises a composition as described herein. In some embodiments, the sample reservoir comprises (i) the recombinant nucleic acid primer; (ii) a recombinase; (iii) a polymerase; (iv) a target nucleic acid; (v) a crowding agent; (vi) one or more primers, and (vi) a single stranded DNA binding protein (SSB). In some embodiments, the sample preparation reservoir further comprises one or more types of signaling agents with different detectable potential for different types of target or amplified nucleic acids. For example, the signaling agents can be a redox dye of differing redox potential for each of the target or amplified nucleic acid. In some embodiments, signals are sensed by the sensor(s) in response to a substrate reacting with the signaling agents localized over the sensor. The reactions of each substrate-signaling agent will differ based on the potential of the signaling agents for the particular type of target nucleic acid.

In some embodiments, a scanning technique such as cyclic voltammetry (CV), square wave voltammetry, or amperometry may be used to probe the localized sample over the sensor(s) for the presence and/or quantity of a nucleic acid target generated in response to nucleic acid targets being introduced in the sample and or any internal control reactions. Such techniques could be used to detect multiple target analyte types including multiple amplified nucleic acid types per sensor to allow for multiplexing on each sensor or different sensors. For example, if there exist three different redox dyes that can be detected at different redox potentials during CV scanning or square wave voltammetry or linear sweep over the known potential range (e.g. −0.6 V to 0.5 V), then there can be three analytes measured per sensor. If there are three sensors, then nine targets could be analyzed within the analysis channel having three sensors. In some embodiments, the device comprises a memory as described in WO 2018/140540. Instructions for executing the scanning technique may be stored in memory coupled to the processor of the reader such that the reader processes the electrical signals sensed by the sensor(s) of the cartridge that are transmitted to the reader, e.g., via respective electrical connectors. For example, the processor of the reader may direct one or more electrodes of the one or more sensors in the cartridge to apply a voltage sweep. The different types of signaling agents may respond to different voltages during the voltage sweep. The response for each type of signaling agent may be used to determine the presence, absence, and/or quantity of the corresponding target analyte.

In some embodiments, isothermal amplification product can proceed simultaneously such that targets of the nucleic acid variety (RNA, DNA) can be measured by combining various affinity and signal molecules and a combination of the above methods.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Original Invasion Oligonucleotide Primer Resulted in Non-Specific Amplification Products

### Overview

The isothermal amplification chemistry discussed herein relies on three primers: a forward primer, a reverse primer, and a novel or improved recombinant nucleic acid primer (oligonucleotide, Invasion Oligonucleotide, or “IO primer”). The IO primer is an integral part of the isothermal amplification process. Together with the recombinase UuvsX, the IO primer mediates strand invasion of the target double stranded DNA (dsDNA) amplicon. The invasion step is a necessary step in the isothermal amplification process because it creates the accessibility required for binding of the forward and/or reverse primers and polymerase to the target double stranded DNA, and thus leads to exponential amplification of the amplicon.

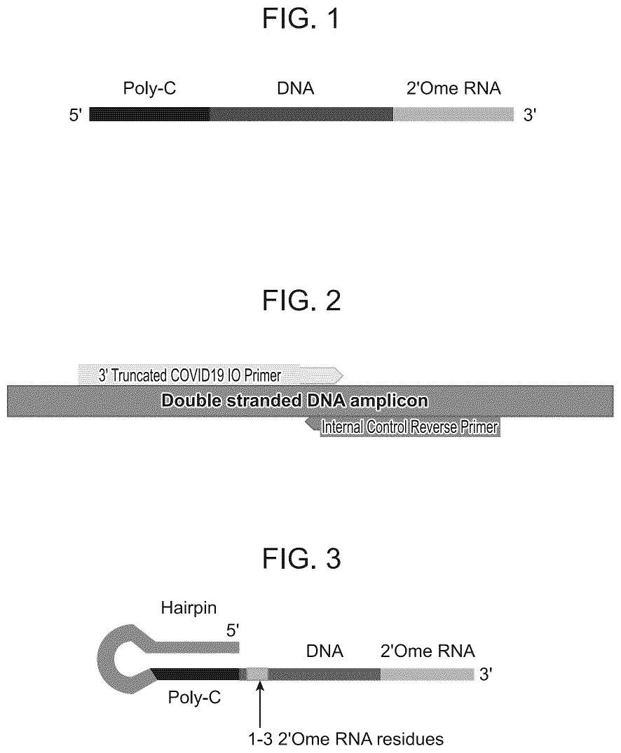

A potential pitfall was observed in the original design of the IO primer (FIG. 1). Experimental evidence showed that the IO primer could itself function as template for the forward and/or reverse primers and polymerase during the amplification process. This phenomenon led to false positive results at problematically high rates in numerous test, for example, the COVID-19 PCR test. Moreover, this problem was exacerbated when the IO primer was used in the multiplexed assay system where the multiplicity of the assay was increased, and/or additional primers was required. Under these circumstances, the probability of non-specific primer-to-primer interactions generally increased. For these reasons, it was concluded that the original design of the IO primer limits the utility of the IO primer for clinical applications, such as for example the COVID-19 PCR Test.

Applicant accordingly hypothesized that the high rate of false positive results when using the original IO primer could be prevented by redesigning the IO primer in either one of three ways: (1) using modified DNA or RNA bases within the IO primer, (2) by introducing secondary structures into the IO primer, or (3) by a combination of the two approaches. The present disclosure provides an optimal design for an IO primer for use in a multiplex assay that exhibits low background characteristics, while maintaining exceptional functionality for template-dependent amplification.

### IO Primer Design

The unmodified, base case of the IO primer design included no secondary structure. As shown in FIG. 1, the original IO primer was about 39-61 bases in length and contained three 3 sub-regions. These three sub-regions, listed from 5′ to 3′ were: (1) a poly-dC sub-region with about 7-13 bases in length; (2) a DNA sub-region that was homologous to the target nucleic acid region and was about 25-35 bases in length; and (3) a methylated RNA sub-region that is also homologous to the target nucleic acid region (length: 7-13 bases).

Next-generation sequencing of amplification products obtained with the original IO primer (also referred to as the recombinant nucleic acid primer; or nonfunctional primer) indicated that the IO primer could function as a substrate for forward and reverse primer and polymerase; and/or as an amplification primer for the amplification reaction (e.g., amplification process). Following the performance of next generation sequencing on multiple amplification products of finished amplification reactions that used the original IO primer, a set of non-specific sequence reads was observed. The presence of these anomalous sequence reads suggested that the IO primer itself could occasionally function as a primer for amplification rather than functioning merely in the invasion of the double stranded nucleic acid target as contemplated by the design. The greatest evidence for this phenomenon was observed with the COVID-19 amplification products. As shown in FIG. 2, the presence of hybrid sequence reads that contained sequences derived from both the COVID-19 IO primer and an internal reverse primer (Internal Control Reverse Primer) was observed. Thus, during the amplification reaction, the COVID-19 10 primer and the Internal Control Reverse primer both unexpectedly aligned with some of the target nucleic acid to generate hybrid amplification products that were observed in the sequencing of products from COVID19-negative reactions.

Furthermore, some sequence reads suggested a mechanism where the internal control reverse primer could bind to and “prime” the amplification (transcription) of the reverse complement of the COVID19 IO primer. The Applicant hypothesized that, the binding of the 3′ end of the forward or reverse primer within the target amplification region (DNA region) of the IO primer could be transcribed by the polymerase to create the reverse complement of the IO. This in turn could lead to exponential non-specific amplification. Such non-specific amplification had generated false positive results in the COVID-19 test.

### Example 2: Evaluation of a Novel Recombinant Nucleic Acid Primer Designs for Isothermal Amplication

Given the above observation, the Applicant opted to include DNA or RNA bases within the IO primer that would not be expected to serve as template for polymerization. The original IO primer was modified to include either 1) an oxidized dG DNA base (oxo-dG) within the DNA sub-region, or 2) inclusion of 2-3 methylated RNA bases within the DNA sub-region.

Additionally, strategies in which the IO primer's structure was modified were also considered. It was hypothesized that by sequestering the poly-dC sub-region of the original IO primer, the suitability of the IO primer to function as a template for polymerization might be reduced. Towards this end, two separate approaches were tested. First, the IO primer was modified to add a short poly-dG oligonucleotide to limit the accessibility of the IO primer's poly-dC sub-region. Second, a 5′ hairpin structure was introduced into the IO primer itself. A combination of these two modifications was also considered where the poly-dC sub-region would function as part of the stem structure.

Each of the above design modifications was tested on the Cue Health Inc cartridge platform, using the company's COVID-19 test as the underlying system. Cue COVID-19 is a biplex test. This test combined primers for amplifying a target that is present in human samples (an internal control template) and primers for amplifying the the SARS-COV-2 virus (assay target). Using this system, the rate of false positive assay results was determined.

To assess the false positive rate (FPR), each IO condition was tested with the addition of only the internal control template in the form of contrived nasal matrix (i.e., no target template added). The results are summarized in Table 1 below. When the original IO primer was used without modification, the False Positive Rate was about 5.1% (196 Negative Tests vs 10 False Positives). When the 5′ hairpin structure was added to the original IO primer, the False Positive Rate was reduced from 5.1% to 1.3% (232 Negative Tests vs 3 False Positives). When the original IO primer was modified to include the 5′ hairpin structure and 2′OME sites, the False Positive Rate was reduced from 5.1% to 0.3% (294 Negative Tests vs 1 False Positive).

TABLE 1
Aggregated cartridge results for different IO primer Modifications.

| | | | | Number of | Number | False |
|---|---|---|---|---|---|---|
| COVID-19 | Nucleotide | Structure | Cartridge | Negative | of False | Positive |
| IO number | composition | changes | Builds | Tests | Positives | Rate |
| 046 | Inclusion of | Addition of | 11359, | 299 | 4 | 1.3% |
| | oxo-dG site | polyG | 11360, | | | |
| | | oligonucleotide | 11383 | | | |
| 053 | Inclusion of | Addition of | 11389, | 197 | 3 | 1.5% |
| | 2′OME sites | polyG | 11396 | | | |
| | | oligonucleotide | | | | |
| 060 | none | Inclusion of 5′ | 11358, | 232 | 3 | 1.3% |
| | | hairpin | 11395, | | | |
| | | structure | 11397 | | | |
| 061 | Inclusion of | Inclusion of 5′ | 11458, | 294 | 1 | 0.3% |
| | 2′OME sites | hairpin | 11473, | | | |
| | | structure* | 11474 | | | |
| control | none | none | 11382, | 196 | 10 | 5.1% |
| | | | 11450 | | | |

Modified IO primers were tested with a COVID19-negative contrived nasal matrix. A few invalids and canceled tests were excluded from the analysis.
*In this condition, hairpin structure were also used for the internal control IO primer.

Similar results were obtained when the original IO primer was modified to contain a polyG oligonucleotide and 2′OME sites or the oxo-dG site. For instance, when the original IO primer was modified to contain a polyG oligonucleotide and 2′OME sites, the the False Positive Rate was reduced from 5.1% to 1.5% (197 Negative Tests vs 3 False Positives). When the original IO primer was modified to contain a polyG oligonucleotide and the oxo-dG site, the the False Positive Rate was reduced from 5.1% to 1.3% (299 Negative Tests vs 4 False Positives).

Overall, the modified condition that implemented inclusion of extra 2′OME sites and the 5′ hairpin structure was the most successful in reducing the occurrence of false positives compared to the control condition (0.3% vs 5.1%).

### Example 3: The Improved IO Primer Reduced False Positive Rate for COVID-19 Test

To determine whether the improved IO primer design could reduce false positive rates for COVID-19 test, each IO modification was tested in amplification reactions with a COVID-19 RNA template added. As shown in Table 2 below, the modified IO primer promoted the successful amplification of target SARS-COV-2 virus genes. In most conditions tested, no false negative result was observed. Two conditions showed 1 false results. These were statistically significant because of the low n value.

TABLE 2
Aggregated cartridge results for different IO primer modifications

| | | | | Number of | |
|---|---|---|---|---|---|
| | | | | COVID19 + | |
| COVID19 | | | | RNA | Number of |
| IO Primer | Base | Structure | Cartridge | Samples | False |
| number | composition | changes | Builds | Tested | Negatives |
| 046 | Inclusion of | Addition of | 11359, | 29 | 1 |
| | oxo-dG site | polyG | 11360, | | |
| | | oligonucleotide | 11383 | | |
| 053 | Inclusion of | Addition of | 11389, | 19 | 0 |
| | 2′OME sites | polyG | 11396 | | |
| | | oligonucleotide | | | |
| 060 | none | Inclusion of 5′ | 11358, | 30 | 0 |
| | | hairpin structure | 11395, | | |
| | | | 11397 | | |
| 061 | Inclusion of | Inclusion of 5′ | 11458, | 30 | 1 |
| | 2′OME sites | hairpin | 11473, | | |
| | | structure* | 11474 | | |
| control | none | none | 11382, | 20 | 0 |
| | | | 11450 | | |

Tested with a COVID19 RNA template (60 copies per test). A few invalids and canceled tests were excluded from the analysis.
*In this condition, hairpin structure was also used for the internal control IO primer.

Overall, the detection was consistent across all conditions, with each resulting in either 0 or 1 false negatives. The modified condition that implemented inclusion of extra 2′OME sites and the 5′ hairpin structure was similarly the most successful in reducing the occurrence of false positives compared to the control condition.

The new design for IO primer, which is optimized for low false positive rate (FPR) in multiplex assay (biplex, triplex, 4-plex conditions) contains about five-subregions as shown in FIG. 3. Listed from 5′ to 3′, the sub-region are (1) 5′ hairpin structure; (2) a a poly-dC sub-region (length: 7-13 bases), (3) a first methylated RNA sub-region comprising 1-4 bases; (4) a DNA sub-region that is homologous to the target (length: 25-35 bases), and (5) a second methylated RNA sub-region that is homologous to the target (length: 7-13 bases). The methylated RNA regions comprise 2′-O-methyl RNA (2′ Ome)

This novel recombinant nucleic acid primer design is resistant to non-specific amplification shown in FIG. 2. This property is key (essential) for the isothermal amplification chemistry to perform well in a clinical setting using the multiplex assay contemplated by the present disclosure. In particular, this novel recombinant nucleic acid primer design was shown to greatly improve the specificity and sensitivity of multiplexed (2-plex, 3-plex, 4-plex, etc.) assays with the compositions and methods described herein.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A recombinant nucleic acid primer (oligonucleotide) comprising:
(i) a 5′ hairpin region, wherein the 5′ hairpin region comprises a 5′ hairpin loop region and a stem region; and
(ii) a target binding region that is complementary to a sequence of a target nucleic acid;
wherein the target binding region comprises at least one modified nucleoside (base) to prevent amplification of the recombinant nucleic acid primer by a polymerase;
wherein the at least one modified base that inhibits amplification by the polymerase is located at one or more of: (i) a 3′-end; (ii) a 5′-end; or (iii) within the internal region of the target binding region to inhibit amplification by the polymerase.

2. The recombinant nucleic acid primer of claim 1,
(i) wherein the at least one modified nucleoside (base) is selected from 5-nitroindole; 8-oxo-2′-deoxyguanosine (8-oxo-dG); 8-oxo7,8-dihydro-2′-deoxyguanosine; 8-oxo-deoxyadenosine (8-oxo-dA); 5-hydroxymethyl deoxycytosine (5-hydroxymethyl-dC); 5′-dimethoxytrityl-N-benzoyl-3′-deoxyadenosine,2′-succinoyl-long chain alkylamino-CPG (3′-dA-CPG); 5′-dimethoxytrityl-N-dimethylformamidine-3′-deoxyguanosine,2′-succinoyl-long chain alkylamino-CPG (3′-dG-CPG); 5′-dimethoxytrityl-N-benzoyl-3′-deoxyCytosine,2′-succinoyl-long chain alkylamino-CPG (3′-dC-CPG); 5′-dimethoxytrityl-3′-deoxythymidine,2′-succinoyl-long chain alkylamino-CPG (3′-dT-CPG); 3′-deoxyadenosine; cytosine arabinoside; inverted dT; 2′-O-methyl RNA nucleotide; 2′-O-methyladenosine (2′-OMe-A); 2′-O-methylcytosine (2′-OMe-C); 2′-O-methylguanosine (2′-OMe-G); 2′-O-methyluridine (2′-OMe-U); inosine, or a modified base that blocks 5′ to 3′ polymerase amplification; or
(ii) wherein the at least one modified nucleoside is a di-deoxy-nucleotide selected from dideoxycytidine (ddC); 2′-3′-dideoxycytidine (2′,3′ dideoxy-C); 5′-Dimethoxytrityl-N-succinoyl-long chain alkylamino-CPG, 2′,3′-deoxycytosine (2′,3′-ddC-CPG); 2′-3′-dideoxyadenosine (2′,3′ dideoxy-A); or 5′-dimethoxytrityl-N-succinoyl-long chain alkylamino-CPG, 2′,3′-deoxyadnosine (2′,3′-ddA-CPG); or
(iii) wherein the target binding region comprises a base modified with a C3 spacer amidite (DMT-1,3-propanediol); a C3 spacer phosphoramidite; a C3 spacer; a C6 spacer; hexanediol; a triethylene glycol spacer (spacer 9); a 18-atom hexa-ethyleneglycol spacer (spacer 18); 1′,2′-Dideoxyribose (dSpacer); a C6 amine; a peptide nucleic acid (PNA); a locked nucleic acid (LNA); or a 1-Dimethoxytrityloxy-propanediol-3-succinoyl)-long chain alkylamino-CPG (3′-Spacer-C3-CPG).

3. (canceled)

4. The recombinant nucleic acid primer of claim 1, wherein:
(i) the 3′-end and the 5′-end of the target binding region comprise one or more modified bases that are the same or different;
(ii) the 3′-end and the internal region of the target binding region comprise the one or more modified bases that are the same or different;
(iii) the 5′-end and the internal region of the target binding region comprise the one or more modified bases that are the same or different; or
(iv) the 3′-end, the 5′-end, and the internal region of the target binding region comprise the one or more modified bases, and wherein at least two of the 3′-end modification, the 5′-end modification, and the internal modification are the same or different.

5. The recombinant nucleic acid primer of claim 1, wherein the modified base is a 2′-O-Methyl RNA (2′OME) nucleotide; optionally, wherein a region comprising at least one 2′OME nucleotides is a methylated RNA region.

6. The recombinant nucleic acid primer of claim 5, wherein the target binding region further comprises a first methylated RNA region and a second methylated RNA region.

7. The recombinant nucleic acid primer of claim 6, wherein:
(i) the first methylated RNA region comprises at least one 2′OME nucleotide, or at least two 2′OME nucleotides, or at least three 2′OME nucleotides, or at least four 2′OME nucleotides, or at least about five 2′OME nucleotides; and/or
(ii) the first methylated RNA region is located at the 5′-end of the target binding region of the recombinant nucleic acid primer; and/or
(iii) the second methylated RNA region is located at the 3′-end of the target binding region; and/or
(iv) the second methylated RNA region comprises at least 7 to at least 13 contiguous nucleotides; and/or
(v) all the complementary nucleic acid residues in the target binding region comprise 2′OME nucleotides.

8. (canceled)

9. A recombinant nucleic acid primer (oligonucleotide) comprising:
(i) a 5′ hairpin region, the hairpin region comprises a 5′ hairpin loop region and a stem region;
(ii) a first methylated RNA region that is complementary to a sequence of a target nucleic acid;
(iii) a target binding region that is complementary to a sequence of the target nucleic acid; and
(iv) a second methylated RNA region that is complementary to a sequence of the target nucleic acid.

10. (canceled)

11. The recombinant nucleic acid primer of claim 9, wherein the 5′ hairpin loop region is about 25 to about 65 nucleotides in length.

12. (canceled)

13. The recombinant nucleic acid primer of claim 12,
wherein the 5′ hairpin loop region comprises an adjacent portion to the stem region and a non-adjacent portion; and
wherein the non-adjacent portion is 5′ to the adjacent portion and the non-adjacent portion of the hairpin loop region hybridizes to the stem region; and wherein the non-adjacent portion of the hairpin loop region comprises a polydeoxyguanylic acid (poly-dG) sequence that is complementary to a polydeoxycytidylic acid (poly-dC) sequence of the stem region; or the non-adjacent portion of the hairpin loop region comprises a polydeoxycytidylic acid (poly-dC) sequence that is complementary to a polydeoxyguanylic acid (poly-dG) sequence of the stem region.

14. The recombinant nucleic acid primer of claim 9, wherein the stem region comprises a polydeoxycytidylic acid (poly-dC) region; and/or wherein the stem region comprises at least about 7-13 contiguous dC bases.

15. The recombinant nucleic acid primer of claim 9, wherein:
(i) the recombinant nucleic acid primer is not extended by a polymerase, thereby rendering the recombinant nucleic acid primer resistant to amplification by a polymerase; and/or
(ii) the recombinant nucleic acid primer does not comprise a 3′-end inverted nucleotide modification; and/or
(iii) the recombinant nucleic acid primer is not a template for amplification, thereby resistant to non-specific amplification caused by the activity of a forward and/or reverse primers binding; and/or
(iv) the first methylated RNA or the second methylated region comprises 2′-O-methyl RNA (2′OME) nucleotides; and/or
(v) the first methylated RNA region comprises at least about one 2′OME nucleotide, or at least about two 2′OME nucleotides, or at least about three 2′OME nucleotides, or at least about four 2′OME nucleotides, or at least about 2′OME nucleotides.

16. The recombinant nucleic acid primer of claim 9, wherein the recombinant nucleic acid primer comprises one or more additional nucleoside modification,
(i) wherein the one or more additional modification is selected from 5-nitroindole; 8-oxo-2′-deoxyguanosine (8-oxo-dG); 8-oxo7,8-dihydro-2′-deoxyguanosine; 8-oxo-deoxyadenosine (8-oxo-dA); 5-hydroxymethyl deoxycytosine (5-hydroxymethyl-dC); 5′-dimethoxytrityl-N-benzoyl-3′-deoxyadenosine,2′-succinoyl-long chain alkylamino-CPG (3′-dA-CPG); 5′-dimethoxytrityl-N-dimethylformamidine-3′-deoxyguanosine,2′-succinoyl-long chain alkylamino-CPG (3′-dG-CPG); 5′-dimethoxytrityl-N-benzoyl-3′-deoxyCytosine,2′-succinoyl-long chain alkylamino-CPG (3′-dC-CPG); 5′-dimethoxytrityl-3′-deoxythymidine,2′-succinoyl-long chain alkylamino-CPG (3′-dT-CPG); 3′-deoxyadenosine; cytosine arabinoside; inverted dT; inosine, or a modified base that blocks 5′ to 3′ polymerase amplification; or
(ii) wherein the one or more additional nucleoside modification is a di-deoxy-nucleotide selected from dideoxycytidine (ddC); 2′-3′-dideoxycytidine (2′,3′ dideoxy-C); 5′-Dimethoxytrityl-N-succinoyl-long chain alkylamino-CPG, 2′,3′-deoxycytosine (2′,3′-ddC-CPG); 2′-3′-dideoxyadenosine (2′,3′ dideoxy-A); or 5′-dimethoxytrityl-N-succinoyl-long chain alkylamino-CPG, 2′,3′-deoxyadnosine (2′,3′-ddA-CPG); or
(iii) wherein the one or more additional nucleoside modification is a nucleoside modified with a C3 spacer amidite (DMT-1,3-propanediol); a C3 spacer phosphoramidite; a C3 spacer; a C6 spacer; hexanediol; a triethylene glycol spacer (spacer 9); a 18-atom hexa-ethyleneglycol spacer (spacer 18); 1′,2′-Dideoxyribose (dSpacer); a C6 amine; a peptide nucleic acid (PNA); a locked nucleic acid (LNA); or a 1-Dimethoxytrityloxy-propanediol-3-succinoyl)-long chain alkylamino-CPG (3′-Spacer-C3-CPG).

17.-19. (canceled)

20. The recombinant nucleic acid primer of claim 15, wherein:
(i) the second methylated RNA region is located at the 3′-end of the recombinant nucleic acid primer;
(ii) the second methylated RNA region comprises at least about 7 to at least about 13 contiguous nucleotides; and
(iii) all the complementary nucleic acid residues are replaced with 2′OME nucleotides.

21.-24. (canceled)

25. A recombinant nucleic acid primer comprising:
(i) a 5′ hairpin region comprising:
a 5′ hairpin loop region that is about 25 to about 65 nucleotides in length; and stem region comprising a polydeoxycytidylic acid (poly-dC) region and/or having at least about 7-13 contiguous dC bases;
(ii) a first methylated RNA region that is complementary to a sequence of a target nucleic acid comprising at least about one to at least about five 2′-methyl RNA (2′OME) nucleotides;
(iii) a target binding region that is complementary to a sequence of the target nucleic acid, and
(iv) a second methylated RNA region that is complementary to a sequence of the target nucleic acid comprising at least about seven to at least about thirteen 2′OME nucleotides;
wherein, the recombinant nucleic acid primer is not a template for amplification;
wherein, the recombinant nucleic acid primer significantly enhanced the specificity and sensitivity of a multiplexed assay when compared to a recombinant nucleic acid primer comprising only the 5′ hairpin region, the first methylated RNA region, or the second methylated RNA region; and
wherein the specificity and sensitivity of multiplexed assay is based on the occurrence of false positive results.

26. The recombinant nucleic acid primer of claim 25, wherein:
(i) the target nucleic acid is a nucleic acid molecule from an infectious agent selected from a coronaviridae virus, a respiratory syncytial virus (RSV), a polio virus, a West Nile virus, a Chikungunya virus, an Ebola virus, a Lassa virus, a Dengue virus, a SARS coronavirus, a Middle East Respiratory Syndrome (MERS) coronavirus, a Junin virus, a hepatitis C virus, a hepatitis B virus, an Influenza A virus, an Influenza B virus, an Influenza C virus, a vaccinia virus, a variola virus, a polyomavirus, a Pox virus, a Herpes virus, a cytomegalovirus (CMV), a human immunodeficiency virus, a JC virus, a JC polyomavirus (JCV), a BK polyomavirus (BKV), a Simian virus 40 (SV40), a Monkeypox virus, a Marburg virus, a Bunyavirus, an arenavirus, an alphavirus, or a flavivirus; or
(ii) the target nucleic acid is from a coronaviridae virus selected from SARS-COV-1, SARS-COV-2, MERS-COV, β-coronaviruses, HCoV-OC43, HCoVHKU1, HCoV-NL63, or HCoV-229E.

27. (canceled)

28. A nucleic acid amplification process comprising the recombinant nucleic acid primer of claim 25.

29. (canceled)

30. The nucleic acid amplification process of claim 28, wherein the nucleic acid amplification process is a homologous recombination enzyme directed isothermal amplification comprising a recombinase enzyme; and wherein the process comprises the steps of:
(i) contacting a recombinase with the recombinant nucleic acid primer to form an oligo-recombinase complex;
(ii) contacting the oligo-recombinase complex to the complementary double stranded target nucleic acid under conditions that allow the oligo-recombinase complex to invade the double stranded target nucleic acid, wherein the oligo-recombinase complex invasion of the double stranded target nucleic acid denatures the region of the double stranded target nucleic acid that is complementary to the recombinant nucleic acid primer;
(iii) admixing a forward primer, a reverse primer, a polymerase, and deoxynucleotide triphosphates (dNTPs) to the process under conditions that allow the forward and reverse primers to bind to their respective complementary regions on the double stranded target nucleic acid, and to allow the polymerase to extend the 3′ end of the forward and reverse primers with dNTPs to generate a first and second amplified double-stranded nucleic acid; and optionally
(iv) continuing the amplification process through repetition of (i) and (iii) until a desired degree of amplification product is produced.

31. The nucleic acid amplification process of claim 30, wherein;
(i) the nucleic acid is amplified in the absence of a recombinase loading factor;
(ii) the nucleic acid is amplified in the presence of single stranded DNA binding molecule (SSB);
(iii) the recombinase is selected from E. coli RecA, a UvsX from any bacteriophage species, T4 UvsX, Rb69 UvsX, T2 UvsX, T6 UvsX, Aeh1 UvsX, KvP40 UvsX, or any derivatives or any combinations thereof;
(iv) the polymerase is selected from E. coli Pol I, Bacillus subtilis Pol I large fragment (Bsu polymerase), Moloney Murine Leukemia Virus (MuMLV) reverse transcriptase, Staphylococcus aureus Pol I, or a combination thereof; and/or
(v) the recombinant nucleic acid primer, the target nucleic acid molecule, the forward and reverse primers, the polymerase, and dNTPs are added simultaneously to the nucleic acid amplification process.

32. The nucleic acid amplification process of claim 31 wherein:
(i) the SSB is selected from E. coli SSB protein, a gp32 protein from any bacteriophage; a gp32 protein derived from a myoviridae bacteriophage, a T4 gp32 protein, Rb69 gp32 protein, or derivatives thereof, or
(ii) the SSB does not bind or binds weakly to the recombinant nucleic acid primer, thereby enhancing the formation of a recombinase-recombinant nucleic acid primer complex, and negating the need of a recombinase loading factor during amplification; or
(iii) the recombinase and the SSB are from the same species; or wherein the recombinase and the SSB are from different species; or
(iv) the recombinase and the SSB are from the same bacteriophage species; or
(v) the recombinase and the SSB are from different bacteriophage species.

33.-38. (canceled)

39. A composition comprising the recombinant nucleic acid primer of claim 25.

40. The composition of claim 39, wherein the composition comprises:
(i) the recombinant nucleic acid primer;
(ii) a recombinase;
(iii) a polymerase;
(iv) a target nucleic acid;
(v) a crowding agent; and
(vi) a single stranded DNA binding protein (SSB).

41. The composition of claim 40, wherein:
(i) the recombinase and the SSB are from the same bacteriophage species; or wherein the recombinase and the SSB are from different bacteriophage species;
(ii) the recombinase and the SSB are derived from a myoviridae bacteriophage, a T4 bacteriophage, Rb69 bacteriophage, T2 bacteriophage, T6 bacteriophage, Aeh1 bacteriophage, KvP40 bacteriophage, or derivatives thereof;
(iii) the composition further comprises dNTPs;
(iv) the composition is dried, freeze-dried, lyophilized powder, a pellet, or a bead;
(v) the composition is lyophilized in a reagent ball;
(vi) wherein the composition is reconstituted with trehalose; and/or
(vii) the composition comprises one or more primer set, wherein each primer set comprises a forward primer and a reverse primer, and each primer set amplifies a different target nucleic acid.

42.-48. (canceled)

49. An amplification device comprising the recombinant nucleic acid primer of claim 25.

50-53. (canceled)
